SEQUENCE LISTING (4 sequences)

SEQ ID NO: 1 - DNA, 21 bp, Artificial Sequence
  Primer
gaaccaggtcacagaaaaagg

SEQ ID NO: 2 - DNA, 22 bp, Artificial Sequence
  Primer
ctgcaagtatttccctcgtagg

SEQ ID NO: 3 - DNA, 20 bp, Artificial Sequence
  Primer
cacgaaaatacttgagagcc

SEQ ID NO: 4 - DNA, 22 bp, Artificial Sequence
  Primer
tctctaccccagaatcagcacc

(19) United States
(12) Patent Application Publication
(10) Pub. No.: US 2006/0166360 A1
(43) Pub. Date: Jul. 27, 2006

(54) Compositions, solutions, and methods used for transplantation

(75) Inventors: Francois Berthiaume, Cambridge, MA (US); Martin Yarmush, Newton, MA (US); Yasuji Mokuno, Toyohashi (JP)

Correspondence Address: CLARK & ELBING LLP, 101 FEDERAL STREET, BOSTON, MA, 02110, US

(73) Assignee: The General Hospital Corporation, Boston, MA (US)

(21) Appl. No.: 10/531,001

(22) PCT Filed: Oct. 17, 2003
(86) PCT No.: PCT/US03/33068

§ 371 Date: Nov. 10, 2005

Related U.S. Application Data
(60) Provisional application No. 60/419,375, filed on Oct. 18, 2002.

(51) Int. Cl. C12N 5/08

(52) U.S. Cl. 435/366

(57) ABSTRACT

This invention discloses a method for reducing the intracellular lipid storage material of a cell, tissue, or organ for transplantation and features solutions, methods and kits that induce the metabolic elimination of lipid storage in a cell, tissue, or organ. In one exemplary approach, the process involves contacting a cell, tissue, or organ with a perfusate solution that include catabolic hormones and amino acids, at physiological conditions, to increase lipid export and lipid oxidation. If desired, the cell, tissue, or organ of the invention may also be heat shock preconditioned. The invention can be used to prepare, recondition, or store a cell, tissue, or organ for transplantation by increasing tolerance to ischemia-reperfusion and cold-preservation related injury.

### BACKGROUND OF THE INVENTION

In general, the present invention relates to cell, tissue, and organ transplantation.

Currently, a major limitation of clinical transplantation is the persistent shortage of organs, which results in an extensive number of patients being placed on wait-lists. Furthermore, a large proportion of patients die even before a suitable transplant can be found.

In the context of liver transplantation, although the majority of liver donors are cadaveric, living and split liver donor techniques are promising alternatives, yet represent only about 3% of the total number of transplants performed in the United States (Sindhi et al. J Ped. Surg. 34: 107-110, 1999). Furthermore, living donor methods are inherently limited because they represent a significant risk to the donor. Another approach is the use of bioartificial liver support systems, which may provide temporary liver function support and, in cases in which the patient recovers from the acute phase of the disease, may avoid the need for a liver transplant altogether. However, in light of the early stages of development of such strategies, transplantations involving cadaveric organs are likely to remain the mainstay for the treatment of organ dysfunction for the foreseeable future.

Further exacerbating the problem of organ shortage is the fact that a significant proportion of donor livers are steatotic or fatty and as a result, often deemed unacceptable for transplantation purposes. A significant number of donor organs are therefore discarded and eliminated from the donor pool even before transplantation. Although usually asymptomatic, the accumulation of lipid in livers, also known as hepatic steatosis, is the most common single predisposing risk factor for postoperative liver failure and accordingly, approximately 65% of livers rejected for transplantation are steatotic (Urena et al., World J. Surg. 22: 837-844, 1998). In fact, it is noteworthy that no single other liver pathology is as prevalent as steatosis and is associated with such a negative impact on the current shortage of donor livers.

Indeed, data from animal models suggest that steatotic livers are far more susceptible to ischemia-reperfusion (I/R) related damage than so-called lean livers. In this respect, I/R causes necrosis and apoptosis of hepatocytes and endothelial cells through the generation of oxygen reactive species and the disruption of the microvasculature, ultimately leading to hepatic failure. Studies on the effect of cold storage of liver followed by rewarming and perfusion also show more extensive damage in fatty livers and a reduced “safe” preservation time before transplantation. In the context of liver transplantation, lipid accumulation in the liver also impairs certain key liver functions namely glucose production and cytochrome p450 detoxification activity (Gupta et al., Am. J. Physiol. 278:E985-E991, 2000; Leclercq et al., Biochem. Biophys. Res. Commun. 268: 337-344, 2000).

Furthermore, livers with mild to moderate steatosis, which are considered marginally acceptable, have a lower graft survival rate (76% vs. 89% for lean livers) at four months post-transplantation. In addition, patients receiving steatotic livers have a mere 77% survival rate at two years post-transplantation in comparison to a 91% survival in patients receiving nonsteatotic livers.

It is therefore clear that methods that salvage or recondition donor livers discarded because of severe steatosis or that increase the success rate of transplanted steatotic livers would significantly reduce the number of patient deaths and help bridge the gap that exists between supply and demand in liver transplantation.

### SUMMARY OF THE INVENTION

As is described in greater detail herein, the present invention provides methods and compositions to prepare a donor cell, tissue, or organ for transplantation into a recipient involving the metabolic reduction of intracellular lipid storage in the tissue or organ. It is useful because it provides for an efficient means to rapidly remove excess lipid storage from virtually any potential source of donor material (such as a cell, tissue, or organ) which is deemed unacceptable for transplantation due to its high fat content. In this particular respect, the present invention is particularly useful to recondition steatotic organs for transplantation, for example. If desired, heat shock preconditioning of the cell, tissue, or organ may also be used for example, to increase the overall ability of the cell, tissue, or organ to withstand ischemia-reperfusion injury. Overall, the present invention has important applications to transplantation because it significantly increases the pool size of available donor material and, as a result, alleviates the current severe shortage of such material, including donor livers. This, in turn, translates into a reduction in the number of patients on the liver transplant waiting list and the number of patients dying before a suitable transplant is found.

Accordingly, in one aspect, the invention features a method for preparing a donor cell, tissue, or organ for transplantation into a recipient. This method involves reducing the intracellular lipid storage material of the cell, tissue, or organ. In preferred embodiments, a human liver cell, human liver tissue, or a human liver organ is prepared.

Preferably, the method of reducing intracellular lipid storage material (e.g., a triglyceride, cholesterol, cholesterol ester, or phospholipid) includes contacting the cell, tissue, or organ with a solution (such as the defatting solution described herein) that increases oxidation of a lipid; increases export of a lipid from the cell, tissue, or organ; or both. In preferred embodiments, the method results in reducing an ischemia-reperfusion injury in the cell, tissue, or organ upon transplantation into a recipient or results in reducing a cold-preservation-related injury in the cell, tissue, or organ upon transplantation into a recipient. In other preferred embodiments, the method reconditions a steatotic cell, tissue, or organ.

If desired, heat shock may also be induced in the cell, tissue or organ of the invention. Heat shock may result from increasing the temperature of the cell, tissue, or organ by at least 1° C. for a period of at least one minute. For example, the temperature may be increased for a period ranging between one minute and one hour, preferably between 1 minute and 30 minutes, and more preferably between 1 minute and 15 minutes. Desirably, the temperature of the cell, tissue, or organ is increased to a range between 37° C. and 50° C., preferably between 38° C. and 45° C., more preferably between 40° C. and 43° C., and most preferably between 42° C. and 43° C.

According to this invention, the increase in temperature may result from heating the whole body or, alternatively, may result from heating a localized area of the donor cell, tissue, or organ. The heating may be mediated by placing the cell, tissue, or organ in a solution (e.g., a defatting solution or saline that has been heated to 42° C.) that induces heat shock; by perfusing the tissue or organ with a solution that induces heat shock; or by warming the blood percolating the localized area in which the cell, tissue, or organ is located. The increase in temperature may also result from heating the cell, tissue, or organ ex vivo. In general, heating may be mediated by microwave or ultrasound treatment.

Alternatively, heat shock may be induced by contacting the cell, tissue, or organ with an agent that increases the expression of at least one heat shock protein. For example, the cell, tissue, or organ may be contacted with an agent such as cobalt protoporphyrin or geranylgeranylacetone. Optionally, the cell, tissue, or organ is administered with a therapeutically effective amount of a heat shock protein or is provided with at least one expression vector containing a nucleic acid sequence encoding a heat shock protein in a therapeutically effective amount. Preferably, the expression of the heat shock protein is increased by at least 10%, 20%, preferably at least 30%, 40%, 50%, 60%, 70%, 80%, 90%, 95%, 100%, or more than 100% relative to an untreated control. Exemplary heat shock proteins include HSP72, HSP70, HO-1, and HSP90.

According to this invention, heat shock preconditioning preferably decreases the proliferation and activation of T cells and decreases the production of inflammatory cytokines (e.g., IL-12, Il-10, IFN γ, and TNF-α.). In this regard, CD4⁺ T cells, for example, produce inflammatory cytokines, activate Kuffpner cells, and recruit neutrophils.

If desired, the cell, tissue, or organ may be contacted with a composition containing gadolinium chloride (GdCl₃) or an agent that inhibits the T cell proliferation, T cell activation, or both. Such an agent may include, for example, cyclosporine A (CyA) and FK506.

The cell, tissue, or organ that has been preconditioned (defatted, heat shock preconditioned, or both) according to this invention is preferably transplanted between 3 to 48 hours, between 6 to 48 hours, or 24 hours after being prepared. If the donor material is not used for transplantation, the donor cell, tissue, or organ may be stored, preferably at 4° C.

In another aspect, the invention features a solution (e.g., a defatting solution) for reducing intracellular lipid storage material of a donor cell, tissue, or organ for transplantation into a recipient; this solution includes a catabolic hormone and an amino acid. In preferred embodiments, the catabolic hormone of the solution increases intracellular lipid oxidation; lipid export; or both. Exemplary catabolic hormones include glucagon, epinephrine, growth hormone, hepatocyte growth factor, leptin, adiponectin, metformin, thyroid hormone, or a glucocorticoid hormone (such as a hydrocortisone, a cortisol, a corticosterone, or dexamethasone). In still other preferred embodiments, an amino acid (such as alanine or glutamine) is required for the synthesis of an apolipoprotein. In yet other preferred embodiments, the solution further includes an anti-oxidant or an oxygen carrier. Exemplary anti-oxidants include N-acetyl-cysteine, glutathione, allopurinol, S-adenosyl-L-methionine (a precursor of glutathione), polyphenols (found, for example, in green tea), free iron scavengers (e.g., deferoxamine), adenosine, or inhibitors of inducible nitric oxide synthase (iNOS) (e.g., N(G)-nitro-L-arginine methyl ester and aminoguanidine) and exemplary oxygen carriers include hemoglobin or a perfluorocarbon. If desired, the solution optionally includes a component that provides oncotic pressure.

In preferred embodiments, the solution includes: from 50 mM to 150 mM sodium ion; from 0.4 mM to 4 mM potassium ion; from 0 mM to 50 mM phosphate ion; from 0 mM to 44 mM bicarbonate ion; from 0.19 mM to 5 mM calcium ion; from 0.081 mM to 5 mM magnesium ion; from 0.2 mM to 2.4 mM alanine; from 0.2 mM to 10 mM glutamine; from 50 pg/mL to 1000 pg/mL glucagon; from 100 pg/mL to 2500 pg/mL epinephrine; from 50 ng/mL to 1500 ng/mL hydrocortisone; and from 30 g/mL to 120 g/mL hydroxyethyl starch.

In still other preferred embodiments, the solution includes: 116 mM sodium ion; 2.3 mM potassium ion; 1.0 mM sodium phosphate (monobasic); 26 mM sodium bicarbonate; 1.9 mM calcium ion; 0.81 mM magnesium ion; 0.48 mM alanine; 2.00 mM glutamine; 100 pg/mL glucagon; 250 pg/mL epinephrine; 150 ng/mL hydrocortisone; and 60.0 g/mL hydroxyethyl starch.

Preferably, the solution is heated to a temperature of 25° C. to 45° C., preferably 25° C. to 43° C., even more preferably 42° C. to 43° C. or 37° C.; is exposed to 20 to 100% O₂, such as 95% O₂; is exposed to 0 to 10% CO₂, such as 5% CO₂; and has a pH of 6.5 to 7.8, such as a pH of 7.4. Optionally, the solution further contains an agent that increases the expression of at least one heat shock protein in a cell, tissue, or organ, such as cobalt protoporphyrin or geranylgeranylacetone.

In still another aspect, the invention features a method for preparing a donor cell, tissue, or organ (including steatotic cells, tissues, or organs) for transplantation into a recipient that includes contacting the donor cell, tissue, or organ with any of the aforementioned solutions. Preferably, the donor cell, tissue, or organ is contacted for at least 10 minutes, 1 hour, 6 hours, 24 hours, or 48 hours.

Additionally, the invention features a method of storing or preserving a donor cell, tissue, or organ for transplantation into a recipient. This method includes contacting the donor cell, tissue, or organ with any of the aforementioned solutions.

The invention further features kits for preparing or storing a donor cell, tissue, or organ for transplantation into a recipient (including kits for preconditioning steatotic cells, tissues, or organs), the kit including a solution for reducing intracellular lipid storage material of the donor cell, tissue, or organ and instructions for using the solution(s) provided in the kit. Optionally, the solution within the kit further contains an agent that increases the expression of at least one heat shock protein in a cell, tissue, or organ, such as cobalt protoporphyrin or geranylgeranylacetone.

The invention further provides a device for preparing a cell, tissue, or organ having excessive fat content for transplantation into a recipient by inducing heat shock in the cell, tissue, or organ. Desirably, such a device contains any of the solutions of the invention, such as a solution for reducing intracellular lipid storage material of a cell, tissue, or organ as described herein. According to this invention, the induction of heat shock may occur in vivo or ex vivo. For example, the device of the invention may increase the temperature of the tissue or organ in a localized area by the emission of ultrasound or microwaves, for example. Alternatively, the device of the invention may have a heat exchanger that allows the cell, tissue, or organ to be contacted with a solution (e.g., defatting solution, saline, or blood) that has been heated and that in turn induces heat shock in the cells of the donor material. Preferably, the device contains a heat exchanger that heats the cell, tissue, or organ to both 37° C. and 42° C. Accordingly, the cell, tissue, or organ being prepared using this device would be defatted and heat shocked, either simultaneously or sequentially. Such an exemplary device is shown in FIG. 1B.

In another aspect, the invention features a cell, tissue, or organ prepared according to any one of the aforementioned methods involving the reduction of intracellular lipid storage material, heat shock preconditioning, or both, and therefore includes isolated defatted donor cells, tissues, or organs that may be used for transplantation into a recipient.

In a final aspect, the invention features a method of transplanting a cell, tissue, or organ, the method including (a) providing any of the aforementioned defatted cells, tissues, or organs; and (b) transplanting such a cell, tissue, or organ into a recipient.

By “lipid storage material” is meant any of a Variety of cellular substances that are soluble in nonpolar organic solvents. Such material includes, without limitation, triglycerides, cholesterol, cholesterol esters, free fatty acids, and phospholipids.

By “reducing intracellular lipid storage material” is meant decreasing an amount of lipid storage material in a cell, tissue, or organ by inducing catabolic metabolism of the lipid storage material by increasing lipid export, lipid oxidation, or both from the cell, tissue, or organ. Typically, the intracellular lipid storage material of a donor cell, tissue, or organ is measured relative to the intracellular lipid storage content of a control cell, tissue, or organ. In preferred embodiments, the lipid storage material of a donor cell, tissue, or organ is reduced by at least 20% (and preferably 30% or 40%) as compared to the lipid storage material of a control cell, tissue, or organ. In other preferred embodiments, the lipid storage material is reduced by at least 50%, 60%, and more preferably reduced by 75%, 80%, 85%, or even 90% of the level of a control; with at least a 95% reduction in lipid storage material as compared to a control being most preferred. The level of lipid storage material is measured using conventional methods, such as those described herein. A reduction in the intracellular lipid storage material of a cell, tissue, or organ is referred to as defatting.

By “induce heat shock” is meant to elicit in a cell, tissue, or organ a response characteristic of the cell's, tissue's, or organ's natural response to elevated temperatures. Typically, induction of heat shock promotes the ability of a cell, tissue, or organ of the invention to withstand ischemia-reperfusion induced damage. According to this invention, heat shock induces the expression of various proteins including heat shock proteins, such as HSP72, HSP70, HO-1, and HSP90. The expression of heat shock proteins may be increased by at least 10%, 20%, preferably at least 30%, 40%, 50%, 60%, 70%, 80%, 90%, 95%, 100%, or even more than 100% relative to such expression in a cell, tissue, or organ in which heat shock has not been induced. Typically, heat shock induction also decreases the proliferation and activation of T cells within the tissue or organ and decreases the production of inflammatory cytokines (e.g., IL-12, Il-10, IFN γ, and TNF α). Preferably, T cell proliferation or activation, or alternatively, the production of inflammatory cytokines is decreased by at least 10%, 20%, 30%, 40%, 50%, 60%, 70%, 80%, 90%, 100% or even more than 100% relative to such proliferation and activation, or alternatively such production, in a cell, tissue, or organ in which heat shock has not been induced. Typically, heat shock is induced by increasing the temperature of the cell, tissue, or organ to a temperature ranging between 37° C. to 50° C., preferably between 38° C. and 45° C., more preferably between 40° C. and 43° C., and even more preferably between 42° C. and 43° C. The temperature of the cell, tissue, or organ may be increased using any method known in the art. Such temperature may be increased, for example, by contacting the cell, tissue, or organ with a solution that has been heated, or alternatively, using ultrasound or microwaves. Optionally, the cell, tissue, or organ may be provided with the heat shock protein or proteins by any method known in the art, including protein microinjection or transfection.

By “ischemia-reperfusion related injury” is meant any damage, including loss of viability, caused to a donor cell, tissue, or organ subsequent to a decrease in the availability of oxygen followed by a sudden increase in oxygen levels. Ischemic or hypoxic conditions for the purposes of the present invention are typically caused by (1) surgical procedures, which require temporary blood flow arrest, including for example liver resection and vascular reconstruction, and (2) storage of the cell, tissue, or organ in the absence of a continuous supply of oxygen. Such conditions allow for the generation of inflammatory mediators, reactive oxygen species, and nitric oxide, as well as the infiltration of neutrophils, which can severely damage cells, tissues, and organs. The length of time necessary for ischemia-related damage is tissue-dependent, and certain cells, tissues, or organs may be more susceptible to hypoxic donations as a result of their high-energy demands.

By “cold-preservation related injury” is meant any damage caused to the cell, tissue, or organ caused by the storage of a cell, tissue, or organ in hypothermic conditions for transplantation purposes. As an example, under hypothermic conditions, phospholipids forming the lipid bilayer of the cellular membranes undergo a phase change leading to a reduction in fluidity. As a result of this phase change, the cell fails to utilize oxygen as efficiently, in a situation analogous to anoxic conditions.

By “anti-oxidant” is meant any agent that scavenges reactive oxygen species, which are generated in instances in which oxygen tension is increased. Changes in oxygen tension may result from a transition from anoxic to normoxic conditions, or from normoxic to supraphysiological oxygen tension. Examples of anti-oxidants include but are not limited to N-acetyl-cysteine, glutathione, allopurinol, S-adenosyl-L-methionine (a precursor of glutathione), polyphenols (e.g., in green tea), free iron scavengers (e.g., deferoxamine), adenosine, or inhibitors of inducible nitric oxide synthase (iNOS) (e.g., N(G)-nitro-L-arginine methyl ester and aminoguanidine), cyclodextrin, superoxide dismutase (SOD), catalase, chlorpromazine, and prostacyclin.

By “reconditioning a cell, tissue, or organ for transplantation” is meant restoring a cell, tissue, or organ, which is deemed unacceptable for transplantation, into a transplantable form.

Although the most widely tested method of organ preconditioning is ischemic preconditioning (induced by clamping major feeding vessels of an organ), such methods may have at best a negligible effect on the survival of transplanted steatotic livers, which are more likely to manifest ischemic injury in comparison with normal lean livers. In contrast to the prior art, the present invention is particularly useful for the preconditioning of steatotic cells, tissues, and organs and is therefore advantageous for several reasons: (1) it will increase the donor pool size, as severely steatotic organs (e.g., livers) are usually discarded; (2) it will improve the outcome of patients who receive organ transplants with mild to moderate steatosis; (3) it will provide a similar approach for a variety of organ systems prone to steatosis during obesity, such as pancreatic β cells and cardiomyocytes; (4) it will provide methods for preventing or limiting hepatic fibrosis, as hepatic steatosis often precedes fibrosis in degenerative liver diseases; and (5) it will further optimize organ preservation techniques and exploit the potential of long-term warm perfusion preservation techniques. Furthermore, the metabolic preconditioning regimens of the invention that reduce the lipid load and modulate the redox state of cells (e.g., liver cells) will reduce the impact of I/R and prolong the preservation time of donor livers.

Other features and advantages of the invention will be apparent from the following Detailed Description, the drawings, and the claims.

### BRIEF DESCRIPTION OF THE DRAWINGS

FIG. 1A shows a schematic diagram of a perfusion apparatus used to defat livers. The liver is immersed in the perfusate solution and perfused via the portal vein at a rate of 4 mL/min/g liver. The perfusate is heated to 37° C. through a heat exchanger and oxygenated by passing through a thin silicone tubular membrane exposed to 95% oxygen and 5% carbon dioxide. A bubble trap is placed immediately before the perfusate enters the liver.

FIG. 1B shows a schematic diagram of a perfusion apparatus used to induce heat shock in a liver. The perfusate solution is heated to 42° C. through a heat exchanger and used to perfuse the liver via the portal vein.

FIGS. 2A-2D show the morphological appearance of cultured hepatocytes after 7 days of plasma exposure. FIG. 2E shows a graph of the intracellular triglyceride levels in hepatocytes for conditions shown in FIGS. 2A-2D. Statistical differences were determined using ANOVA with Tukey's post hoc test (n=11).

FIG. 3 shows the effect of defatting medium on cultured hepatocyte appearance after 2 days of defatting.

FIGS. 4A and 4B show the release of lactate dehydrogenase by cultured hepatocytes after I/R at 37° C. FIG. 4A shows the effect of hypoxic time before reoxygenation in steatotic and normal “lean” hepatocytes. FIG. 4B shows the effect of defatting time on the response of steatotic hepatocytes to I/R.

FIGS. 5A and 5B show the release of lactate dehydrogenase (LDH) by cultured hepatocytes in response to 12 hours of storage at 4° C. followed by rewarming at 37° C. FIG. 5A shows LDH activity after 12 hours in University of Wisconsin (UW) solution at 4° C. and 12 more hours at 37° C. in medium. FIG. 5B shows the effect of defatting time on the response of steatotic hepatocytes to cold storage followed by rewarming.

FIG. 6 shows the proportion of cytochrome c detected in the cytosolic fraction of hepatocytes after 12 hours of storage at 4° C. followed by rewarming at 37° C. Cytosolic cytochrome c is normalized to total (cytosolic+mitochondrial) cytochrome c.

FIGS. 7A and 7B show the effect of hepatocyte island size and steatosis on hepatocyte viability after I/R. FIG. 7A shows intensity of calcein fluorescence per surface area over hepatocyte islands at various time points during I/R of steatotic hepatocytes co-cultured with nonparenchymal cells. FIG. 7B shows calcein fluorescence per surface area over hepatocyte islands at the 4 hour time point (1 hour of no flow followed by 3 hours of flow).

FIGS. 8A and 8B show that rats fed a choline and methionine-deficient diet (CMDD) developed fatty livers. FIG. 9A shows the kinetics of hepatic triglyceride (TG) accumulation in rats fed a CMDD for up to 6 weeks. FIG. 9B shows the restoration of the hepatic TG content to normal levels upon return of CMDD animals to a regular diet.

FIG. 9 shows that defatting makes fatty donor livers suitable for transplantation. Survival curves for rats receiving donor livers are shown. Livers were stored for 6 hours in UW solution prior to transplantation. CMDD refers to fatty liver recipients. CMDD+RF 3d or 7d refers to recipients receiving donor livers from CMDD fed rats followed by refeeding (RF) with a normal diet for 3 or 7 days, respectively.

FIGS. 10A and 10B show the effect of amino acids in the perfusate on liver triglyceride content after 3 hours of warn perfusion (panel A) and the effect of perfusion time on liver triglyceride content using amino acid-containing perfusate (panel B). Fatty livers from CMDD fed rats for 6 weeks were perfused at 37° C. After 3 hours of perfusion, the remaining TG content is in the normal range (˜10 mg/g liver).

FIG. 11A is a series of bar graphs showing the levels of HSP72 as measured by ELISA, in livers harvested before heat shock preconditioning (HPc) (pre) or between 3 and 72 hours after HPc. Data shown are for HPc-fatty livers (n=6), sham HPc-fatty livers (n=3), and HPc-normal livers (n=7). Bars represent mean±SD. * P<0.05 compared to “pre” levels. #P<0.05 compared to levels at three hours. $P<0.05 compared to levels at 6 hours. &P<0.05 compared to levels at 12 hours. §P<0.05 compared to levels at 24 hours.

FIG. 11B is a series of immunoblots showing the protein expression of HSP72, HO-1, and HSP90 in fatty livers. Data shown are representative of three rats in the HPc and one rat in the sham HPc groups.

FIG. 11C is a series of bar graphs representing the quantification of protein bands shown in FIG. 12B.

FIGS. 12A-12D are a series of bar graphs showing the effect of heat preconditioning (HPc) on the levels of hepatic enzymes and inflammatory cytokines induced by the transplantation of fatty livers. Donor fatty livers were harvested 24 hours after HPc (▴) or sham HPc (∘), preserved in cold UW solution for 10 hours, and then transplanted into syngeneic animals. ALT (FIG. 12A) and AST (FIG. 12B) activities in the serum of the recipient, as well as serum TNF-α (FIG. 12C) and IL-10 (FIG. 12D) levels, are shown as measured by ELISA. Data shown represent the mean±SD for 6 rats. *P<0.05 between groups. **P<0.01 between groups.

FIGS. 13A-13H are a series of photographs showing the effect of heat preconditioning on fatty liver transplantation. HPc prevents hemorrhage and confluent hepatocellular necrosis in fatty livers. The transplanted livers from HPc (FIGS. 13B, 13D, 13F, and 13H) or sham-HPc (FIGS. 13A, 13C, 13E, and 13G) donors were harvested 3 hours (FIGS. 13A, 13B, 13C, and 13D) or 24 hours (FIGS. 13E, 13F, 13G, AND 13H) after revascularization, and stained by hematoxylin and eosin. Severe congestion, hemorrhagic change (arrows), and areas of confluent hepatocellular necrosis (arrow heads) were seen in the sham-treated group, with significant reduction of these findings seen in the HPc group at all histologic features considered reduction in extent and degree of hemorrhagic injury was the most striking hallmark of the HPc group. Original magnification FIGS. 13A, 13B, 13E, and 13F, 40×; FIGS. 13C, 13D, 13G, and 13H, 120×.

FIG. 14 is a graph showing the effect of heat preconditioning (HPc) on the survival of recipients after fatty liver transplantation. Transplantation of cold preserved (for 10 hours) fatty livers was performed 24 hours after HPc (▴; n=12) or sham HPc (∘; n=12). Also shown are data for transplantation of cold preserved (for 10 hours) normal livers (▪; n=7). Differences among groups: fatty liver HPc vs. fatty liver sham HPc (P<0.005); normal liver vs. fatty liver sham HPc (P<0.01); normal liver vs. fatty liver HPc (not significant).

FIG. 15 is a series of immunoblots showing the protein expression of HSP72 and HO-1 in hepatocytes, CD4⁺ T cells, and CD8⁺ T cells in steatotic rat liver after HPc (n=3), sham HPc (n=3), and GdCl₃ treatment (n=3). Livers were harvested 24 hours after treatment and CD4⁺ and CD8⁺ T cells were separated by flow cytometry. The expression level of HSP72 and HO-1 was analyzed by Western blot. 5 μg of total protein was loaded in each lane and the data is representative of three separate experiments.

FIG. 16 is a graph showing the effect of heat shock preconditioning or GdCl₃ on serum heptic enzyme levels (ALT) after fatty liver transplantation. Donor livers were harvested 24 hours after HPc (n=5), GdCl₃ injection (n=5), or sham HPc (n=5), preserved in cold UW solution for 12 hours and then transplanted. Sera were collected from recipient rat up to 24 hours after hepatic revascularization and measured for levels of ALT activity. Data are representative of 3 separate experiments and show mean±SD for 5 rats. *p<0.05 compared to Sham group. **p<0.01 compared to Sham group.

FIGS. 17A-17F are photographs showing the effect of heat shock preconditioning or GdCl₃ on the morphology of transplanted fatty livers. The transplanted livers from sham-heat shock preconditioned (17A and 17D), heat shock preconditioned (17B and 17E), or GdCl₃ pretreated (17C and 17F) donor were obtained 3 and 24 hours after revascularization, and stained by hematoxylin and eosin staining. Original magnification; 100×.

FIGS. 18A-18C is a series of graphs showing the effect of heat shock preconditioning or GdCl₃ on the level of serum cytokines after fatty liver transplantation. Donor livers were harvested 24 hours after HPc (n=6), GdCl₃ injection (n=6) or sham HPc (n=6), preserved in cold UW solution for 12 hours and then transplanted. Sera were collected from recipient rat up to 24 hours after hepatic revascularization and measured for levels of IL-12p70, TNF-α, and IL-10. Data are representative of three separate experiments and show the mean±SD for 5 rats. *p<0.05 compared to Sham group.

FIG. 19 is a bar graph showing the effect of heat shock preconditioning or GdCl₃ on myeloperoxidase in the liver after fatty liver transplantation. Donor livers were harvested 24 hours after HPc (n=6), GdCl₃ injection (n=6) or sham HPc (n=6), preserved in cold UW solution for 12 hours, and then transplanted. Livers were harvested from recipient rat 3 hours and 24 hours after hepatic revascularization and measured for levels of myeloperoxidase in liver tissues. Data are representative of 2 separate experiments and show the mean±SD for 5 rats. *p<0.05 compared to Sham group.

FIG. 20 is a graph showing the effect of heat shock preconditioning (HPc) or GdCl₃ on the survival of recipient rats after liver transplantation. Donor livers were harvested 24 hours after HPc, GdCl₃ injection, or sham HPc, preserved in cold UW solution for 12 hours, and then transplanted. Survival rate of recipient rats was monitored for up to 1 week after transplantation. *: p<0.01 compared to Sham group. **: p<0.001 compared to Sham group.

FIG. 21A is a series of agarose gel photographs showing the level of mRNA expression of IFN-γ in liver CD4⁺ T cells purified from liver of rats 24 hours after transplantation. Donor livers were harvested 24 hours after HPc, GdCl₃ injection or sham HPc, preserved in cold UW solution for 12 hours, and then transplanted. 24 hours after transplantation, CD4⁺ T cells were purified from liver lymphocytes pooled from 3 rats of each group, and mRNA was isolated for RT-PCR.

FIG. 21B is a graph showing the level of IFN-γ production by liver CD4⁺ T cells (5×10⁵/well) purified from rats 24 hours after transplantation. Cells were incubated in anti-CD3 mAb-coated 96-well plates for 48 hours at 37° C. after which the culture supernatants were collected. Cytokine activity in the culture supernatant was determined for the presence of IFN-γ by ELISA. Data are representative of 3 separate experiments and show mean±SD for 5 rats. *p<0.05 compared to Sham group. **: p<0.001 compared to Sham group.

FIG. 22A is a series of agarose gel photographs showing the level of IFN-γ mRNA in CD4⁺ T cells isolated from transplanted fatty livers following pretreatment with cyclosporin A (CyA) treatment. mRNA expression of purified lymphocytes isolated from liver of rats was determined 24 hours after transplantation. Donor livers were harvested 24 hours after sham HPc, HPc, and GdCl₃ injection, as well as 6 hours after CyA treatment, preserved in cold UW solution for 12 hours and then transplanted. 24 hours after transplantation, CD4⁺ T cells were purified from liver lymphocytes pooled from 3 rats of each group, and mRNA was isolated for RT-PCR.

FIG. 22B is a bar graph showing the level of hepatic enzyme levels in transplanted fatty livers following CyA pretreatment. Donor livers were harvested 24 hours after sham HPc (n=6) and HPc (n=6), and 6 hours after CyA treatment (n=6), preserved in cold UW solution for 12 hours, and then transplanted. Sera were collected from recipient rats up to 24 hours after hepatic revascularization and measured for levels of ALT activity. Data are representative of 3 separate experiments and show mean±SD for 5 rats. *p<0.05 compared to Sham group. **: p<0.001 compared to Sham group.

### DETAILED DESCRIPTION

In general, the present invention provides methods, solutions, and devices for the metabolic preconditioning of a donor cell, tissue, or organ for surgical purposes, including transplantation. These methods involve reducing the intracellular lipid storage material of cells, tissues, or organs thereby increasing their ability to withstand ischemia/reperfusion injuries (I/R), cold-preservation injuries, or both. If desired, heat shock may also be induced in the cells, tissues, or organs of the present invention. Accordingly, the metabolic and heat shock preconditioning methods described herein improve the outcome of virtually any transplant surgical procedures and reduce the risk of postoperative organ dysfunction to a level similar to that observed in nonsteatotic organs (e.g., livers).

Ischemia-Reperfusion (I/R) Injury

Ischemia-reperfusion (I/R) injury is inevitable in complex surgical procedures, such as liver transplantation and liver resection. In this regard, hepatic steatosis is a major risk factor of primary malfunction of graft livers because steatotic livers are especially susceptible to such injury.

Metabolic Preconditioning

Based on our identification of critical branch points of the hepatic metabolic network affected by lipid loading, we hereby provide methods and solutions useful for reducing lipid storage in donor cells, tissues, or organs. To this end, we have shown that two strategies may be used to reduce the lipid load, namely (1) hormonal modulation and (2) amino acid supplementation. With the ultimate goal of using lipid-lowering techniques in the clinic, noninvasive methods for monitoring such “delipidization” processes may also be employed in the methods of the invention. Exemplary monitoring methods include nuclear magnetic resonance (NMR) and positron emission tomography (PET) for quantitatively assessing lipid load and metabolism; furthermore, a judicious choice of probes may also be used alone or simultaneously to monitor the quality of perfusion and the energy status of cells, tissues, or organs.

Optimization of a Metabolic Network

Lipids are typically stored in the liver as triglycerides and are removed by catabolic action. When this occurs, one molecule of triglyceride is broken down into one molecule of glycerol and three molecules of fatty acids, after which fatty acids undergo β-oxidation in the mitochondria to generate reducing equivalents, CO₂, and ketone bodies. Triglycerides can also be removed from the liver by export in the form of lipoproteins.

The methods of the invention involve maximizing the sum of fluxes represented by β-oxidation and triglyceride export. Furthermore, the present methods involve maintaining the intracellular triglyceride synthesis flux to a minimum. These three fluxes are related to each other as well as to the other metabolic fluxes via the stoichiometry of the hepatic metabolic network, which imposes mass balance constraints to the set of possible fluxes.

Optimization of Fluxes

The predicted optimum fluxes are induced experimentally by a combination of mass action effects, for example, by altering amino acid levels in the perfusate or culture medium, and hormones which favor fatty acid oxidation and export of triglycerides, e.g., glucagon, epinephrine, growth hormone, hepatocyte growth factor, thyroid hormone, leptin, adiponectin, metformin, and various glucocorticoid hormones. The steatotic hepatocyte culture system described herein is used in this optimization effort, and the most effective regimen is then utilized in the steatotic perfused liver system. Results from the first studies are analyzed and re-fed into a linear optimization routine in order to generate other predicted optimum perfusate compositions, which are then utilized for treating a donor cell, tissue, or organ. Going through several iterations with this process, the levels of all components of the perfusate may be optimized. Other optimization methods, such as those using empirical simplex algorithms may be used as well.

During the experiments, culture medium/perfusate samples are obtained at regular intervals and the intrahepatic content of triglycerides and glycogen determined as well. Cultured hepatocyte defatting experiments are performed for 24-48 hours and liver perfusions up to 3 hours, which is sufficient to assess the effect of the defatting procedure. Control hepatocytes or livers from littermates are not defatted and instead used to provide the initial values of lipid/glycogen content. Throughout these studies, metabolic flux analyses are performed to characterize the lipid lowering mechanisms, and determine whether the cellular metabolic state returns to that found in normal nonsteatotic livers as the lipid load disappears. To help in the optimization aspects of defatting perfused livers, noninvasive fat measurement methods based on proton chemical shift nuclear magnetic resonance (NMR) imaging and positron emission tomography (PET) using 1-[¹¹C]-3-R,S-methylheptadecanoic acid as a tracer are used to follow the process of delipidization in real time.

Defatting Solutions

Organ preservation and perfusate solutions are known in the art as comprising a base solution that consists of a buffered physiological solution, such as a salt solution or a cell culture-like basal medium, to which is added a variety of defined supplements. In a preferred embodiment, the defatting solution of the present invention also employs such a base solution containing amino acids, ions (e.g., sodium ion, potassium ion, phosphate ion, calcium ion, magnesium ion, and bicarbonate ion), physiologic salts, impermeants, serum proteins and/or factors, and sugars (e.g., glucose). In addition to the components of the base solution, the defatting solution of the present invention contains a novel combination of supplements that can be grouped into at least two component categories. It can be appreciated by those skilled in the art that the components in each category may be substituted with a functionally equivalent compound to achieve the same result. Thus, the following listed species of components in each component category is for purposes of illustration, and not limitation.

A first component category, hormones, comprises a combination of components in a physiologically effective amount, which provide a means to reduce the lipid content in a cell, tissue, or organ by increasing lipid oxidation and lipid export from the cell, tissue, or organ. To insure that this catabolic activity in the cell, tissue, or organ is maintained, conditions characteristic of starvation and thus amenable to lipid reduction are provided. These conditions may include high concentrations of catabolic hormones (e.g., glucagon, epinephrine, growth hormone, hepatocyte growth factor, thyroid hormone, leptin, adiponectin, metformin, or glucocorticoid hormones including for example hydrocortisone, corticosterone, cortisol and dexamethasone) and low concentrations of anabolic hormones (e.g., insulin). The result of using such a combination of hormones simulate conditions of starvation in a mammal and as such, the lipid content of a cell, tissue, or organ is effectively reduced through the oxidation and the export of lipids. The hormones comprise from about 1×10⁻⁶% to about 3×10⁻⁵% by volume (w/v) of the novel combination of supplements, which are added to the base solution in forming the defatting solution of the present invention.

A second component category, amino acids, comprises a combination of components in a physiologically effective amount, which provide a means to supply the building blocks required for the synthesis of apolipoproteins, which are subsequently incorporated into larger lipoproteins. These lipoproteins export triglycerides and other lipids (e.g., cholesterol, cholesterol esters, and phopholipids) outside of the cell, tissue, or organ. Such amino acids added to the defatting solution may include any of the essential nutritional amino acid such as alanine, arginine, aspargine, aspartate, cysteine, glutamate, glutamine, glycine, histidine, isoleucine, leucine, lysine, methionine, phenylalanine, proline, serine, threonine, tryptophan, tyrosine, valine; and a combination thereof. The amino acids comprise from about 0.01% to about 1% by volume of the novel combination of supplements, which are added to the base solution in forming the defatting solution of the present invention.

It will be appreciated by those skilled in the art that components in any one or more of the two component categories can have additional functions desirable for the process according to the present invention. For example, amino acids contained in the defatting solution include cysteine in amounts which, besides functioning as a building block for lipoproteins, also function as antioxidant-preferred free radical scavengers which scavenge toxic free radicals during the flushing and perfusing steps of the process. These toxic free radicals are generated in instances in which oxygen tension is increased (e.g., transition from anoxic to normoxic conditions, or from normoxic to supraphysiological oxygen tension). Other antioxidants, including for example N-acetyl-cysteine, glutathione, allopurinol, S-adenosyl-L-methionine (a precursor of glutathione), polyphenols (e.g., in green tea), free iron scavengers (e.g., deferoxamine), adenosine, or inhibitors of inducible nitric oxide synthase (iNOS) (e.g., N(G)-nitro-L-arginine methyl ester and aminoguanidine), cyclodextrin, superoxide dismutase (SOD), catalase, chlorpromazine, and prostacyclin may be included, or used as functionally equivalent compounds, in the defatting solution of the present invention. If present, such antioxidants comprise from about 0.01% to about 5.00% by volume of the novel combination of supplements, which are added to, and dissolved in, the base solution in forming the defatting solution of the present invention.

In another embodiment of the present invention, the defatting solution may further comprise cytoprotective agents, which can prevent apoptosis of cells resulting from the production of ceramide, a bi-product of lipid accumulation. Such cytoprotective agents can include, for example, membrane-permeable peptidic caspase inhibitors, cyclosporin A, and the inhibitor of ceramide production L-cycloserine. Other agents such as vitamins (e.g., choline chloride, folic acid, myo-inositol, niacinamide, pantothenic acid, pyridoxal HCl, riboflavin, thiamine HCL), ions (e.g., sodium chloride, potassium sulfate, sodium phosphate (monobasic), sodium bicarbonate, calcium chloride, and magnesium sulfate), carbohydrates (e.g., glucose), and pH indicators (e.g., phenol red) may also be included in the defatting solution. Optionally, the defatting solution may also contain agents, which can decrease lipid peroxidation, neutrophil infiltration, microcirculatory alterations, and the release of proinflammatory mediators such as TNF-α. The addition of such agents would provide a means to minimize any damage caused by ischemia-reperfusion injury. Agents which can provide oncotic pressure may also be added to the defatting solution, including, but not limited to, albumin, hydroxyethyl starch, or any high molecular weight polymer.

In another embodiment of the present invention, to avoid the use of supraphysiological oxygen tension and perfusion flow rate, the defatting solution contains one or more oxygen transporting compounds (“oxygen carrying agents”) that function to provide molecular oxygen for oxidative metabolism to the ischemically damaged and injured organ. Such oxygen carrying agents are known to those skilled in the art to include, but not be limited to, hemoglobin, stabilized hemoglobin derivatives (made from hemolyzed human or bovine erythrocytes such as pyridoxylated hemoglobin), polyoxethylene conjugates (PHP), recombinant hemoglobin products, perfluorochemical (PFC) emulsions and/or perfluorochemical microbubbles (collectively referred to as “perfluorochemical”). Such oxygen carrying agents comprise from about 0% to about 50% by volume of the novel combination of supplements, which are added to, and dissolved in, the base solution in forming the defatting solution of the present invention; or about 0% to about 20% of the total defatting solution (v/v).

In a process for preparing the defatting solution according to the present invention, to a base solution is added and dissolved therein a novel combination of supplements that can be grouped in at least two component categories comprising hormones and amino acids. Although the composition of the defatting solution for use with the process according to the present invention can vary by component and component ranges as previously described, a preferred formulation is set forth below in Table 1 for purposes of illustration and not limitation.

The defatting solution thus prepared has an osmolarity >280 mOsm but preferably less than 600 mOsm, and in a preferable range of about 300 mOsm to about 350 mOsm. The pH of the resuscitation solution is typically adjusted to a pH within a pH range of about 6.5 to about 7.8, and preferably in a pH range of 7.3 to 7.45. The defatting solution may also be heated to a temperature of 25 to 40° C., but preferably, is heated to 34 to 39° C. The solution may also be exposed to 20 to 100% O₂ and 0 to 10% CO₂, but preferably 95% O₂ and 5% CO₂.

In still another embodiment, the defatting solution may further include antioxidants, oxygen carrying agents, ions, carbohydrates, vitamins, agents that can provide oncotic pressure and pH indicators as indicated in Table 1.

TABLE 1
Exemplary composition of a perfusate solution for defatting livers.

| Component | Concentration* | |
|---|---|---|
| Salts and Carbohydrates | | |
| Sodium chloride | 116 | |
| Potassium sulfate | 2.3 | |
| Sodium phosphate, monobasic | 1.0 | |
| Sodium bicarbonate | 26 | |
| Calcium chloride | 1.9 | |
| Magnesium sulfate | 0.81 | |
| Glucose | 5.6 | |
| Amino Acids | | |
| Alanine | 0.48 | |
| Arginine | 0.72 | |
| Asparagine | 0.78 | |
| Aspartate | 0.063 | |
| Cysteine | 0.26 | |
| Glutamate | 0.33 | |
| Glutamine | 2.00 | |
| Glycine | 0.38 | |
| Histidine | 0.27 | |
| Isoleucine | 0.40 | |
| Leucine | 0.40 | |
| Lysine | 0.50 | |
| Methionine | 0.10 | |
| Phenylalanine | 0.19 | |
| Proline | 0.42 | |
| Serine | 0.63 | |
| Threonine | 0.40 | |
| Tryptophan | 0.049 | |
| Tyrosine | 0.29 | |
| Valine | 0.39 | |
| Hormones | | |
| Insulin | 20 | μU/mL |
| Glucagon | 100 | pg/mL |
| Epinephrine | 250 | pg/mL |
| Hydrocortisone | 150 | ng/mL |
| Anti-oxidants and | | |
| Cytoprotective Agents | | |
| N-acetyl-cysteine | 2.0 | |
| Adenosine | 5.0 | |
| Glutathione | 3.0 | |
| Allopurinol | 1.0 | |
| Vitamins and Others | | |
| Hydroxyethyl starch | 60.0 | g/mL |
| Choline chloride | 7.1 × 10⁻³ | |
| Folic acid | 2.3 × 10⁻³ | |
| Myo-inositol | 11 × 10⁻³ | |
| Niacinamide | 8.2 × 10⁻³ | |
| Pantothenic acid | 4.2 × 10⁻³ | |
| Pyridoxal HCl | 4.9 × 10⁻³ | |
| Riboflavin | 0.27 × 10⁻³ | |
| Thiamine HCl | 3.0 × 10⁻³ | |
| Phenol red | 31 × 10⁻³ | |

*All values are in mM except otherwise indicated.

Heat Shock Preconditioning

In addition to the metabolic conditioning methods described above, we have also investigated the protective mechanism of heat shock preconditioning (HPc) on recipient survival in fatty liver transplantation. For the purpose of our experiments, we compared the effects of such treatment with gadolinium chloride pretreatment (GdCl₃), and Cyclosporine A pretreatment (CyA) on I/R injury in an experimental choline- and methionine-deficient diet induced rat fatty liver transplantation model.

Our results show that the induction of heat shock by exposing donor rats to brief whole body hyperthermia (10 minutes at 42.5° C.) significantly improved the survival rate post-transplantation in normal rats relative to donor rats that had not been treated (>80% survival after one week vs. <10%). Evaluating the survival of recipients receiving fatty livers at different times following HPc, the protective effect of HPc was most significant when donors were transplanted 3-48 hours after HPc, with the maximal effect seen 6-48 hours after HPc. Histological evaluation 3 and 24 hours after transplantation revealed that HPc significantly reduced hepatic inflammation and hepatocellular necrosis without affecting the steatotic appearance of hepatocytes. We further showed that heat shock preconditioning was concomitant with an induction in heat shock proteins (HSP72, HSP90, and heme oxygenase-1 (HO-1)) in donor livers, with expression levels peaking 12 to 48 hours after HPc.

Attenuation of Cellular Component in I/R Injury by HPc

Experimental I/R injury involves a cascade of events initiated by reactive oxygen intermediates and ultimately resulting in graft invasion by neutrophils and lymphocytes. To this end, membrane-derived compounds (e.g., platelet-activating factor), cytokines (e.g., tumor necrosis factor and macrophage inflammatory protein-2), and adhesion molecules (e.g., the CD18 family, intracellular adhesion molecule-1, and selectins) are thought to depend on the activation of Kupffer cells. Collectively, these factors play a pivotal role in the recruitment and activation of neutrophils.

I/R injury has recently been demonstrated to occur in a biphasic pattern: an initial acute phase characterized by hepatocellular damage (at 3-6 hours) and a subacute phase characterized by massive neutrophil infiltration (at 18-24 hours), in which the activation of CD4⁺ T cells plays a central role.

CD4⁺ T cells are subdivided into at least two subpopulations based on their functional pattern of secreted cytokines, Th1 and Th2. Th1 cells, which secrete IFN-γ, TNF-α and GM-CSF, may represent the best candidates for mediating inflammation. Among the various T cell-secreted cytokines, IFN-γ and TNF-α are known to be potent activator of Kupffer cells and may likely promote local secretion of TNF-α and IL-1, which in turn facilitates the interaction between endothelial cells and neutrophils by activating neutrophils directly or by inducing changes in surface adhesion molecules on endothelial cells. Furthermore, Th1-secreted IFN-γ and GM-CSF may also act directly on neutrophils and enhance their ability to damage liver tissue.

Serum alanine aminoatransferase (ALT), serum cytokines, liver histology, and liver CD4⁺ T cells were next analyzed. As described above, I/R injury in the liver has been demonstrated to occur in a biphasic pattern: an initial acute phase, characterized by hepatocellular damage at 3-6 hours and a subacute phase, characterized by massive neutrophil infiltration at 18-24 hours. Similarly, the liver I/R injury in our model following transplantation demonstrated a biphasic pattern, namely an acute and a subacute phase. While HPc protected transplanted liver against I/R injury both in the acute (3 hours) and the subacute (24 hours) phase, pretreatment with GdCl₃ (a potent inhibitor of Kupffer cell function) only protected I/R injury in the acute phase. However, both HPc and GdCl₃ prevented the serum release of IL-12, TNF-α, and IL-10 produced by Kupffer cells. Kupffer cells are a major source of reactive oxidants and proinflammatory cytokines that promote neutrophil recruitment and adhesion, and eventually lead to organ injury. Thus, our results show that HPc could improve the overall recipient survival rate following transplantation while treatment with GdCl₃ does not.

Our results also demonstrate a key role for CD4+ T cells in liver I/R injury. HPc suppressed the IFN-γ production in liver CD4⁺ T cells 24 hours after transplantation, while GdCl₃ did not. CyA also suppressed the IFN-γ production in liver CD4⁺ T cells and decreased serum ALT levels, an event associated with liver injury. Thus, our results showed that liver CD4⁺ T cells are involved in the cascade leading to the release of cytokines and the development of liver injury. Thus, our results showed that HPc protects from liver I/R injury by modulating the activation of both Kupffer cells and liver T cells in steatotic liver transplantation in rat. Because GdCl₃ pretreatment did not suppress activation of or IFN-γ production by liver T cells 24 hours after transplantation and because GdCl₃ pretreatment did not suppress MPO level of transplanted liver tissue or recipient survival rate after transplantation, T cell involvement may lie proximal to the activation of Kupffer cells. Furthermore, T cells may be critical for the amplification of primary Kupffer cell cytokine responses within the initial phases of injury. Overall, our results indicate that heat shock precondititoning may have great potential for clinical applications by preventing the I/R injury that is associated with steatotic liver transplantation.

Based on the above results, heat shock preconditioning may be used, in addition to metabolic conditioning, to prepare the cells, tissues, and organs of the invention. Desirably, the cells, tissues, and organs have an elevated fat content, and even more desirably, such cells, tissues, and organs are steatotic. HPc may be induced by increasing the temperature of the cell, tissue, or organ of the invention by at least 1° C. for at least one minute. Typically, the temperature is increased for a period ranging between one minute to one hour, preferably between one minute and thirty minutes, and more preferably between one minute and fifteen minutes. The temperature of the cell, tissue, or organ may be increased to a temperature ranging between 37° C. to 50° C., preferably between 38° C. and 45° C., more preferably between 40° C. and 43° C., and even more preferably between 42° C. and 43° C. Such an increase in temperature may be accomplished by any method known in the art. For example, HPc may result from heating the whole body of the donor, or alternatively, may result from heating of the cell, tissue, or organ ex vivo. In this regard, a steatotic liver may be harvested from the donor, heated for a period of 1 minute, placed in cold storage, and then transplanted into a recipient mammal. Furthermore, the cell, tissue, or organ may be heated by localized heating, using microwave or ultrasound treatment for example. Alternatively, HPc may be mediated by warming the blood percolating the localized area of the cell, tissue, or organ of interest. HPc may also be induced by contacting the cell, tissue, or organ with a solution (e.g., defatting solution) that has been heated. Alternatively, the cell, tissue, or organ is contacted with an agent that increases the expression of at least one heat shock protein. Exemplary heat shock proteins include HSP72; HSP70, HSP90, and HO-1. Agents such as cobalt protoporphyrin and geranylgeranylacetone are useful for this purpose. Alternatively, the cell, tissue, or organ of the invention may be provided with a therapeutically effective amount of at least one heat shock protein. In this regard, the heat shock protein may be provided as a recombinant polypepeptide (e.g., by means of mircroinjection) or using an expression vector containing a nucleic acid sequence encoding a heat shock protein (e.g., a plasmid or a viral vector, such as an adenovirus, retrovirus, lentivirus, poxvirus, adeno-associated virus, herpes simplex virus, or vaccinia virus) by any standard method known in the art. Using any of the above methods, the expression of the heat shock protein is increased by at least 10%, 20%, preferably at least 30%, 40%, 50%, 60%, 70%, 80%, 90%, 95%, preferably 99%, more preferably 100%, or even more than 100% relative to an untreated control as measured by any standard method known in the art.

HSPs may protect the cell, tissue, or organ from I/R injury by several mechanisms, namely by providing anti-oxidant functions, by maintaining the patency of hepatic microcirculation, by inhibiting apoptosis in sinusoidal endothelial cells and hepatocytes, or by downregulating inflammation (e.g., by decreasing the production of inflammatory cytokines and by suppressing NF-κB activation and subsequent TNF-α production by Kupffer cells following I/R injury). In this particular regard, our results clearly show that increases in TNF-α and IL-10, observed as early as 3 hours after transplantation in the untreated group, were dramatically reduced by HPc treatment.

The observed reduction in early cellular damage (as shown by the reduction in the levels of ALT and AST) may further reduce the inflammatory stimulus. Accordingly, heat shock preconditioning as taught herein preferably decreases T cell proliferation, T cell activation, or both (e.g., in CD4+ T cells) by at least 10%, 20%, 30%, 40%, 50%, 60%, 70%, 80%, 90%, 95%, preferably 99%, more preferably 100%, or even more than 100% relative to an untreated control. CD4+ T cells produce inflammatory cytokines, activate Kuffpner cells, and recruit neutrophils. As a result of such a reduction, the production of cytokines, such as IL-10, IL-12, IFN-γ, and TNF-α, is decreased. Overall, our data suggests that HPc decreases the cellular damage and the inflammatory response that occurs early after transplantation. If desired, the cell, tissue, or organ that has undergone HPc preconditioning according to the invention may further be contacted with a composition containing GdCl₃ or an agent that inhibits the proliferation, activation, or both of T cells (e.g., cyclosporine A or FK506). Optionally, the cell, tissue, or organ of the invention may be contacted with anti-TNF-α antibodies; FR167653, an agent that suppresses cytokine generation and decreases hepatic IR injury; inhibitors of Kupffer cell activation, adenosine, and antioxidants (e.g., α-tocopherol, lazaroid, and superoxide dismutase).

Donor Transplant Material

A donor cell according to the invention may be obtained from virtually any source, autologous or heterologous, including kidney, heart, liver, lung, intestine, pancreas, bone marrow, and eye. Similarly, a donor tissue or organ includes, without limitation, kidney, heart, liver, lung, intestine, pancreas, bone marrow, and eye.

Regimen/Apparatus/Timing

Cells, tissues, and organs can be defatted by simple incubation with any solution described herein, for example, the solution disclosed in Table 1. Any cell, tissue or organ in which reduction of intracellular lipid material is desirable, including, for example, the liver, the kidney, the pancreas, the heart, the lung, the small bowel, the brain, the eye, or the skin may be contacted or perfused with the defatting solutions disclosed herein.

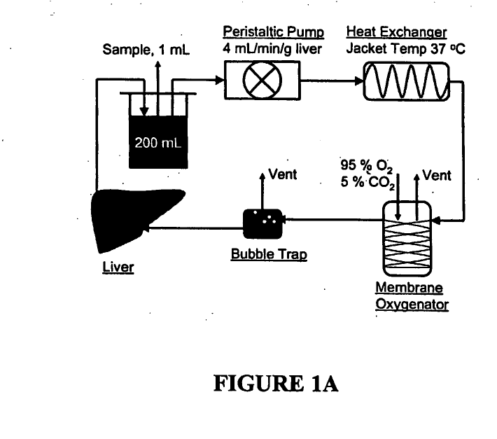

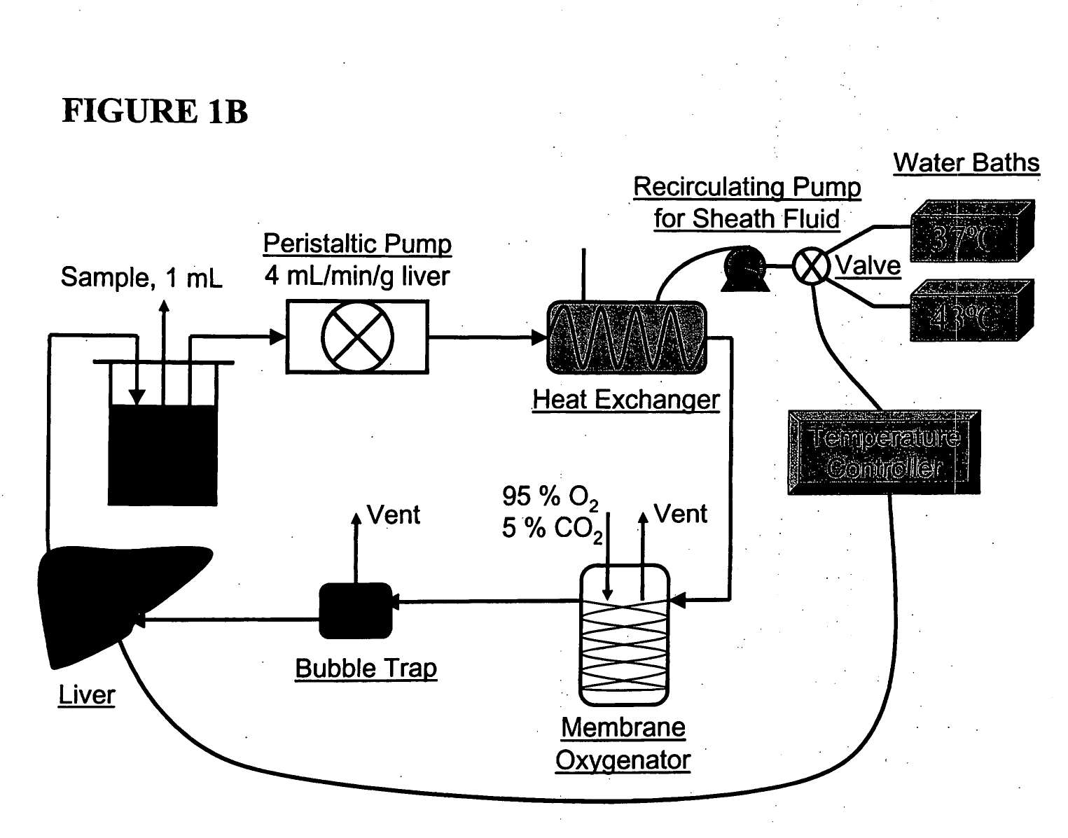

If desired, cells, tissues, or organs are perfused with a defatting solution using the perfusion apparatus shown in FIGS. 1A and 1B. As a specific example, the liver can be immersed in the perfusate solution (preferably the defatting solution described herein) and perfused via the portal vein at a rate of 4 mL/min/g of liver. Perfusion rate can range between 1 mL/min/g to 5 mL/min/g, but preferably, perfusion should take place between 3 mL/min/g to 4 mL/min/g. The perfusate solution is heated to 37° C. (or 42° C. if HPc is desired) through a heat exchanger and oxygenated by passing through a thin silicone tubular membrane exposed to 95% oxygen and 5% carbon dioxide. A bubble trap may be placed immediately before the perfusate enters the liver.

Cells, tissues, and organs can be treated with the defatting solution according to standard methods for a period of time sufficient to enable defatting, including, 10 minutes, 30 minutes, 1 hour, 2 hours, or more than 2 hours. In preferred embodiments, a donor cell, tissue, or organ is treated with a defatting solution for two to three hours.

If desired, heat shock may also be induced in the cell, tissue, or organ having excessive fat content and may be prepared for transplantation using the device of the invention. According to this invention, the device may contain a heat exchanger that increases the temperature of the solution that contacts the tissue or organ. The cell, tissue, or organ may therefore be contacted with a solution (such as blood, saline, and preferably the defatting solution described herein) that has been heated to 42° C. using the device described above (see FIG. 1B). Alternatively, heat shock may be induced in a cell, tissue, or organ using a device that increases temperature in a localized area of the tissue or organ. The cell, tissue, or organ may or may not be in the donor (in vivo or ex vivo, respectively), and the increase in temperature may result from microwaves or ultrasound emitted from the device.

Assessment of Fat Content in Donor Cells, Tissues, or Organs

Fat content of donor cells, tissues, or organs is determined according to standard methods in the art. For example, the cell, tissue, or organ may be examined histologically or biochemically (using a biochemical assay kit) to assess triglyceride content. Alternatively, ¹³³Xenon hepatic retention may also be used as an accurate index for fatty liver quantification (Ahmad et al., J. Nucl. Med. 20: 397-401, 1979; Yeh et al., J. Nucl. Med. 30: 1708-1712, 1989). A less invasive method is based on the fact that the peak resonance frequency of ¹H nuclei of water differs significantly from that of aliphatic carbons (—CH₂—); proton chemical shift magnetic resonance imaging proved to be a sensitive and accurate way to evaluate the localization and quantity of fat deposits in liver and even bone marrow (Rosen et al., Radiology 169: 469-472, 1985; Rosen et al., Radiology 169: 799-804, 1988).

Storage/Preservation

The defatting solutions of the invention can be used to store, preserve, and/or protect cells, tissues, or organs when these materials are brought into contact with the solution. A specific embodiment of the invention is for the preservation or storage of a human liver, or human liver tissue or cells. Another embodiment of the invention is for the preservation of a human heart or human heart tissue or cells. The invention contemplates the use of the defatting solutions to preserve mammalian cells, tissues, organs, or portion thereof. If desired, heat shock may also be induced in the cells, tissues, or organs prior to, or during, storage and preservation. In addition, the solutions can be used to facilitate transplantation of organs, e.g., by perfusion of the organ or tissue during the transplantation procedure. Preferably, the organ or portion thereof is maintained in the appropriate solution at all times.

The defatting solutions of the invention can be used to maintain viability of cells, tissues, or organs during storage, transplantation, or other surgery. The invention includes a method of storing cells, tissues, or organs comprising contacting a donor cell, tissue, or organ, with the solution of the invention, such that the in vivo and/or in vitro viability is prolonged. The solutions permit maintenance of viability of a cell, tissue, or organ (e.g., a liver, heart, or lung) for up to 24 hours. Use of the solutions of the invention results in improved viability.

Kits

The present invention advantageously provides convenient kits for use by practitioners in the art for conveniently preparing a donor cell, tissue, or organ for transplantation into a recipient. In a preferred embodiment, a kit of the invention will provide sterile components suitable for easy use in the surgical environment. A kit of the invention may provide sterile, defatting solution for preparing a donor cell, tissue, or organ for transplantation into a recipient. Generally, such a kit will include a defatting solution or a HPc-inducing solution as described herein in appropriate containers, and optimally packaged with directions for use of the kit. For example, a kit of the invention can provide in an appropriate container or containers: (a) a predetermined amount of at least one defatting solution; (b) if necessary, other reagents; and (c) directions for use of the kit for cell, tissue, or organ treatment or storage.

Transplantation

Once a cell, tissue, or organ is processed using the procedures described herein, such donor material is transplanted into a recipient (e.g., a human) according to standard methods known in the art. Following metabolic preconditioning, HPc, or both, the cell, tissue, or organ may be placed in cold storage and transplanted into the recipient mammal 3 to 48 hours after HPc and preferably between 6 to 48 hours after HPc.

The following experimental examples are provided for the purpose of illustrating the invention, and should not be construed as limiting.

Modulation of Lipid Accumulation in Hepatocytes Cultured in Plasma

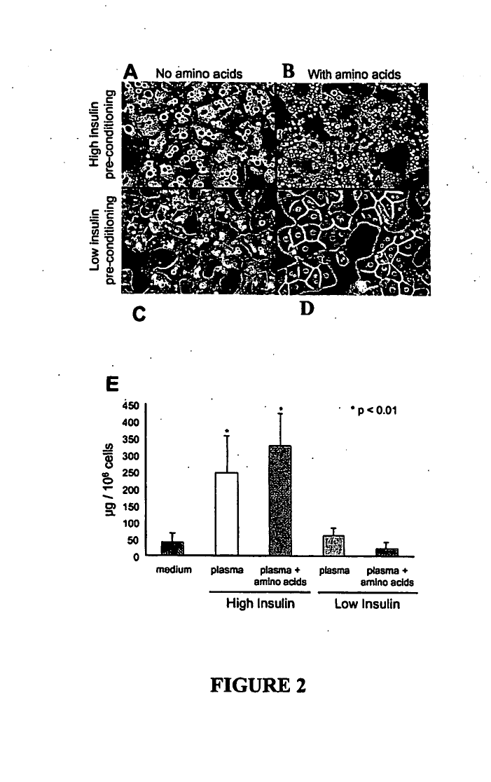

It has previously been shown that collagen-sandwiched adult rat hepatocytes which are seeded and maintained in standard hepatocyte culture medium and then exposed to either rat or human plasma become severely steatotic within 24 hours with a concomitant reduction in liver-specific functions (Matthew et al., Biotechnol. Bioeng. 51: 100-111, 1995; Stefanovich et al., J. Surg. Res. 66: 57-63, 1996). More recently, we found that intracellular accumulation of lipids occurs during exposure to plasma if hepatocytes are cultured in a medium containing high levels of insulin (e.g., similar to that found in standard hepatocyte culture medium or 500 mU/mL) prior to plasma exposure (Chan et al., Biotechnol. Bioeng. 78: 753-760, 2002). On the other hand, hepatocytes cultured in medium containing low insulin levels (50 μU/mL) exhibited little triglyceride accumulation during subsequent plasma exposure. In addition, triglyceride accumulation could be further reduced by direct plasma supplementation with an amino acid cocktail as disclosed in Table 1 (FIG. 2).

We also measured the expression of various liver-specific functions by hepatocytes exposed to plasma. We found that despite the tremendous accumulation of intracellular lipids, amino acid supplementation to the plasma allows hepatocytes to maintain the production of albumin and urea, as well as cytochrome P450 activities to levels similar to or even higher than hepatocytes in standard culture medium (Washizu et al., J. Surg. Res. 93: 237-246, 2000; Washizu et al., Tissue Eng. 6: 497-504, 2000; Washizu et al., Tissue Eng. 7:691-703, 2001). Thus, we concluded that by culturing hepatocytes in high insulin-containing hepatocyte culture medium followed by exposure to plasma supplemented with amino acids, we could obtain steatotic hepatocytes expressing high levels of liver-specific function. Recalling that steatotic livers do not generally show impaired functions in the absence of stressful conditions, these hepatocytes would appear to be a suitable model of steatotic liver.

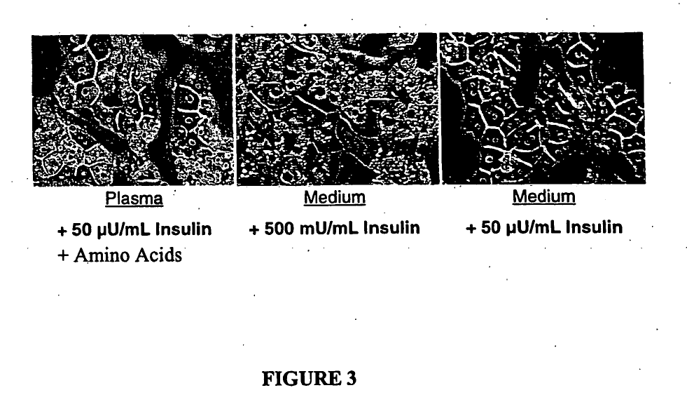

In the next set of experiments, we induced steatosis in hepatocytes by exposing them to high insulin levels followed by plasma for 2 days, and attempted to defat them using the following conditions: plasma supplemented with amino acids and low insulin levels; culture medium containing high insulin (500 mU/mL); culture medium containing low insulin (50 μU/mL) levels. We then measured the fraction of remaining triglycerides after 1 and 2 days of treatment. Low insulin-containing medium almost completely removed intracellular triglycerides (FIG. 3 and Table 2). The triglyceride removal kinetic data in Table 2 was used to calculate a defatting rate for each defatting condition. For this purpose, the fraction of initial triglyceride remaining was plotted as a function of time on a semi-log plot, which yielded linear curves (not shown). The slopes of these lines, which correspond to the first order rate of decay or triglyceride clearance from the cells during defatting, are shown in Table 2 for each defatting condition tested. From these values, we can predict the fraction of intracellular lipid remaining after any treatment time using the simple equation:
Triglyceride Fraction Remaining=100e^{-[RateConstant][TreatmentTime]} (equation 1)

TABLE 2

| | | | 1st | | | |
|---|---|---|---|---|---|---|
| | Initial | | Order | Metabolic Rates for 1^{st} Day of Defatting | | |
| | Triglyceride | | Decay | (μg/10⁶ cells/day) | | |
| “Defatting” | % Remaining* | | Rate Cst | Triglyceride | Triglyceride | Ketone Body |
| Medium | Day 1 | Day 2 | (h⁻¹) | Removal | Secretion | Secretion |
| Plasma, 50 μU/mL | 85 | 62 | 0.010 | 108 | −32 | 130 |
| insulin + amino | | | | | | |
| acids | | | | | | |
| Medium, 500 | 74 | 52 | 0.014 | 170 | 194 | 47 |
| mU/mL insulin | | | | | | |
| Medium, 50 | 30 | 4 | 0.067 | 273 | 384 | 96 |
| μU/mL insulin | | | | | | |

*Initial intracellular triglyceride content was 583 ± 120 μg/10⁶ cells.

Using the low insulin-containing medium, which was the most efficient at defatting, we can estimate that a treatment time of about 10 hours would be sufficient to remove 50% of the intracellular triglycerides, and 28 hours to remove 85%, the latter of which would correspond to normalizing the triglyceride content of a severely steatotic liver. This was a surprising result considering that a limited number of defatting conditions were tried, and it is expected that further optimization of this protocol will significantly reduce these defatting times. It is important to note that liver-specific functions, as determined by the albumin and urea secretion rates, were not reduced during exposure to this medium.

To determine the mechanism of defatting, we measured the triglyceride and ketone body secretion rates in the medium. We found that both of these rates were higher in the low insulin compared to the high insulin-containing medium. In hepatocytes continuously exposed to plasma, there was no net secretion of triglycerides, which probably explains the slower defatting rate. It is interesting to note that the total mass of triglycerides released into the medium exceeded the rate of defatting, especially in the low insulin-containing medium, suggesting that a significant part of the triglycerides released arises from de novo synthesis in hepatocytes. Thus, it is anticipated that addition of drugs which inhibit triglyceride or free fatty acid synthesis (e.g. see Loftus et al., Science 288: 2379-2381, 2000) could significantly accelerate the rate of triglyceride clearance from hepatocytes. We also investigated the effects of leptin and hepatocyte growth factor on the defatting process. In low insulin-containing medium, these agents did not further enhance lipid removal. Some fat-reducing effects were seen in high insulin containing media, albeit not as dramatic as the reduction observed by lowering the insulin concentration.

Response of Steatotic Hepatocytes to Ischemia/Reperfusion

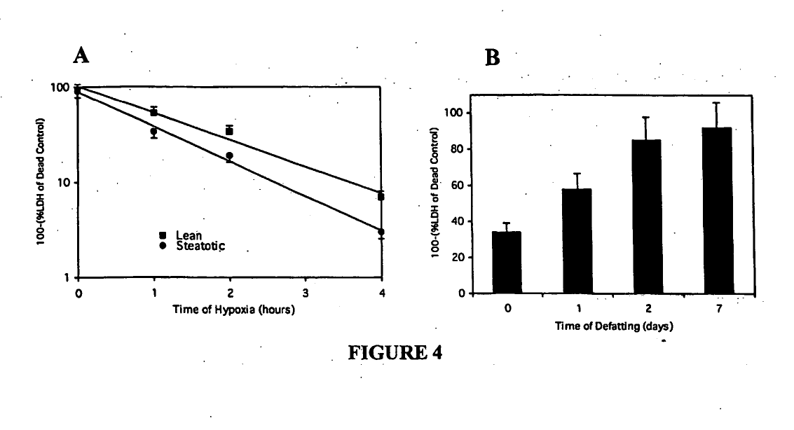

To investigate whether I/R injury correlates with the level of triglyceride loading in hepatocytes, we studied the response of normal and steatotic hepatocytes to I/R. Steatotic hepatocytes were generated by exposure to plasma supplemented with 500 mU/mL insulin and amino acids for 2 days. I/R was induced by switching the cells to an atmosphere containing 90% N₂ and 10% CO₂ for various lengths of time, after which the cells were returned to normoxic conditions. Culture supernatants were harvested 12 hours after restoration of normoxia for the determination of lactate dehydrogenase release, a measure of cell lysis. Lactate dehydrogenase activity in the supernatant was normalized to that of dead controls (hepatocytes subjected to rapid freeze-thaw). We found that steatotic hepatocytes are more sensitive to I/R than lean hepatocytes (FIG. 4A). To determine whether the lipid content at the time of I/R is what determines the sensitivity of cells to I/R, hepatocytes were defatted for different lengths of time prior to I/R. Cell lysis after I/R decreased as a function of defatting time (FIG. 4B).

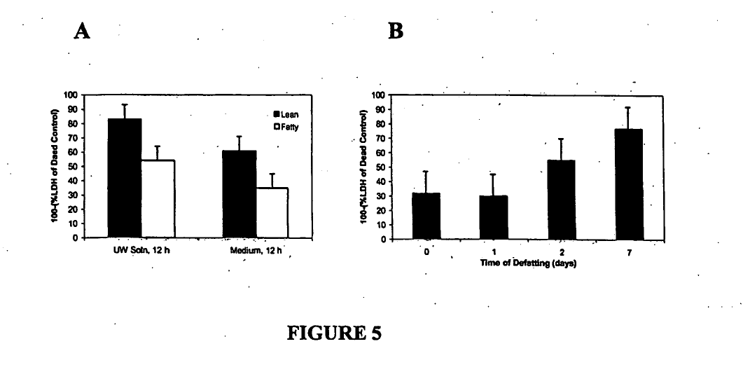

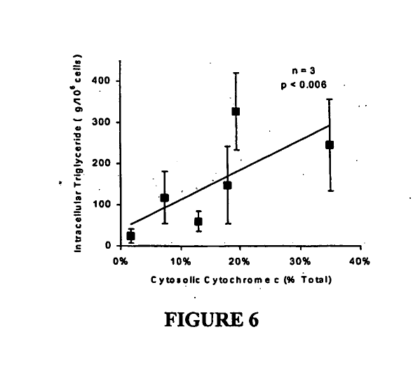

In order to provide additional evidence that the lipid load indeed determines the resistance of cultured hepatocytes to I/R, we investigated the effect of cold storage followed by rewarming on hepatocyte lysis. Hepatocytes were made steatotic by culturing in plasma for 2 days, after which they were incubated in the UW solution at 4° C. for 12 hours. The cells were then returned to standard hepatocyte culture medium at 37° C. for 12 hours, and the release of lactate dehydrogenase in the medium was determined. Consistent with prior observations, lactate dehydrogenase release correlated with the amount of intracellular lipids in the hepatocytes (FIGS. 5A and 5B). As a preliminary assessment of the potential mechanisms of death in this cell culture model, we measured cytochrome c release from the mitochondrial to the cytosolic fraction of the cells, an indicator of apoptosis. Cytochrome c was quantified on Western blots of cytosolic and mitochondrial fractions of hepatocytes subjected to different defatting regimen leading to varying triglyceride content at the time of I/R. We found that cytochrome c release was significantly correlated (p<0.006) with triglyceride storage in hepatocytes (FIG. 6), consistent with the greater extent of cell death shown in FIGS. 5A and 5B.

Effect of Liver Nonparenchymal Cells on the Hepatocyte Response to I/R in Co-Cultures

Since various in vivo studies suggest that Kupffer cells may be activated by I/R and exacerbate the injury (Lichtman and Lermasters, Sem. Liver Dis. 19: 171-187, 1999), we investigated the effect of nonparenchymal cells on the response of steatotic hepatocytes to I/R in micropatterned co-cultures. Hepatocytes were patterned as islands of sizes ranging from 36 to 490 μm on tissue culture dishes using stencil technology described by Folch et al. (J. Biomed. Mater Res. 52: 346-353, 2000; Ann. Rev. Biomed. Eng. 2: 227-256, 2000). The nonparenchymal cell fraction obtained from another liver cell isolation was then seeded on top of the hepatocytes. Nonparenchymal cells only attach to the vacant spaces left in-between the hepatocyte islands. Thus, one can increase direct hepatocyte-nonparenchymal cell interactions by reducing the size of hepatocyte islands, and vice-versa (Bhatia et al., J. Biomed. Mat. Res. 34: 189-199, 1997; Bhatia et al., Biotechnol. Prog. 14: 378-387, 1998; Bhatia et al., J. Biomater. Sci. Polym. Ed. 9:1137-1160, 1998). The cultures were then exposed to plasma supplemented with high insulin levels and amino acids for 2 days to cause steatosis. Five minutes before starting the I/R experiment, 1 μM calcein acetoxymethyl ester was added to the cells for 5 minutes. This compound is specifically retained and converted to brightly fluorescent calcein within viable cells and released upon membrane rupture at the time of cell death.

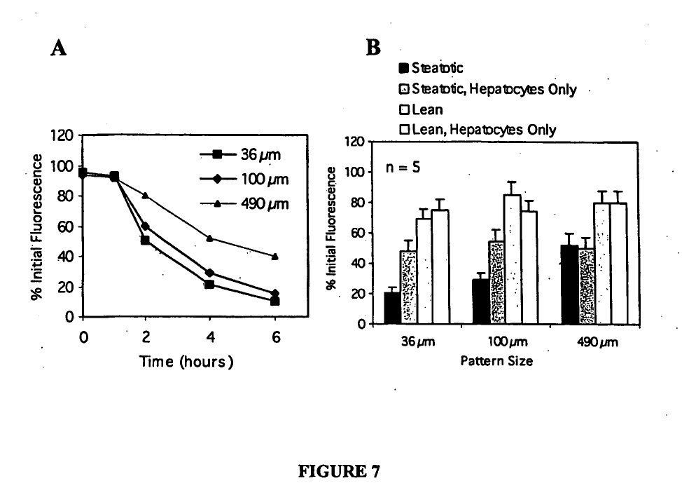

A small flow device made by micro-molding of polydimethylsiloxane as described elsewhere (Folch and Toner, Biotechnol. Prog. 14: 388-392, 1998) was placed on top of the cells to create a mini cell perfusion bioreactor. The bioreactor was perfused with medium saturated with 90% air/10% CO₂ for 1 hour. The flow was then stopped for 1 hour. Because of the low aspect ratio of the flow channel above the cells (1 cm long, 1 cm wide, and 100 μm high), hypoxia occurs inside the flow channel within a few minutes, which mimics the situation in the actual liver when blood flow is stopped. Flow was then restored and cells visualized for an additional 5 hours. The I/R experiment was set up on the temperature-controlled stage of an inverted fluorescence microscope fitted with a digital video camera and image analysis software to quantify the fluorescence intensity distribution of at regular times intervals. Since in these experiments we were primarily interested in hepatocyte viability, the intensity of calcein fluorescence per surface area over hepatocyte islands only was measured, averaged for each island size, and normalized to that measured initially. Hepatocyte viability, based on the fraction of initial calcein fluorescence intensity, decreased as a function of time after reoxygenation and was lower in the smaller hepatocyte islands (FIG. 7A). In addition, hepatocyte viability decreased as a function of hepatocyte island size in co-cultures and was lower in co-cultures than in pure hepatocyte cultures (FIG. 7B). These data strongly support the hypothesis that nonparenchymal cells have deleterious effects on hepatocyte viability after I/R. The data also show that steatotic hepatocytes are more sensitive to I/R than lean hepatocytes, confirming our earlier data based on lactate dehydrogenase release in static cultures.

Non-Invasive Imaging of Hepatic Lipid Metabolism

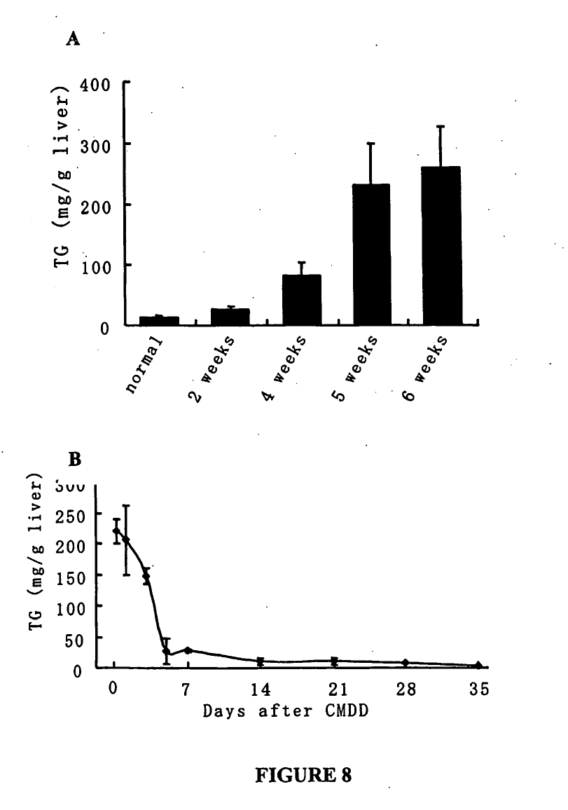

Non-invasive quantitation of hepatic lipid content and metabolism is potentially very useful to optimize and monitor the effect of defatting regimens. Prior studies have shown that proton chemical shift nuclear magnetic resonance (NMR) imaging can provide a quantitative measurement of the liver fat content (Rosen et al., Radiology, 154: 469-472, 1985). In these experiments, rats were either alcohol-fed or received an intraperitoneal injection of ethionine, a protein synthesis inhibitor, to cause lipid accumulation. The NMR signal intensity was directly proportional to the hepatic triglyceride content measured using a biochemical assay (FIGS. 8A and 8B).

This technique is noninvasive and does not require the animal or patient to undergo any particular preparatory procedures, except for the requirement of immobilization, as the imaging time takes about 45 minutes. More recently, we applied the same technique to non-invasively determine fat distribution in bone marrow of human patients (Rosen et al., Radiology, 169: 799-804, 1988). Later on, we found that the distribution of fat determined by this technique is a useful surrogate marker to monitor the severity of Gaucher disease and the efficacy of treatments against acute leukemia (Gerard et al., Radiology, 183:39-46, 1992; Johnson et al., Radiology 182:451-455, 1992). This technique may, if desired, be combined with other techniques to determine microvascular flow distribution and ATP levels in tissue during liver perfusions.

We have also developed methods to determine metabolic fluxes through the tricarboxylic acid and gluconeogenic pathways using ¹³C-NMR spectroscopy and gas chromatography-mass spectroscopy, which we used to investigate metabolic changes in burned rats and patients (Vogt et al., Am. J. Physiol. 266:E1012-1022, 1994; Vogt et al., Am. J. Physiol. 272: C2049-2062, 1997; Yarmush et al., J. Burn Care Rehabil 20: 292-302, 1999). As part of these studies, we recently improved the mathematical formalism used to determine fluxes from ¹³C isotopic distributions by implementing “atom mapping matrices,” which allow one to quickly optimize labeling strategies and adapt the quantitative model for data analysis (Zupke et al., Anal. Biochem. 247: 287-293, 1997). This technology is useful in analyzing metabolic pathways of fatty acid oxidation and metabolism, and independently verify metabolic fluxes obtained with the stoichiometric mass balance model.

Because there are currently no real-time imaging techniques using NMR to study carbon metabolism, positron emission tomography (PET) is typically used to non-invasively monitor regional metabolism in burned patients. For example, previous studies in our laboratory have demonstrated that PET and parallel arterial sampling after bolus injection of L-[methyl-¹¹C]methionine and 1-[¹¹C]-3-R,S-methylheptadecanoic acid can provide less invasive, regional assessments of the protein synthetic rate and fatty acid oxidation rate, respectively, than traditional approaches (Zaknun, J. Nucl. Med. 36: 2062-2068, 1995; Hsu et al., Proc. Natl. Acad. Sci. U.S.A. 93: 1841-1846, 1996). In sum, we have established that PET can be used to study carbon metabolism in healthy human subjects and animals, and that it holds promise for future in vivo, non-invasive studies of the influences of physiological factors and pharmacological manipulations on regional metabolism (Fischman et al., Proc. Natl. Acad. Sci. USA. 27: 12793-12798, 1998).

Defatting of Rat Livers Restores Survival of Recipients

To induce hepatic steatosis in rats, rats (age/weight), prior to surgery, were fed a choline- and methionine-deficient diet (CMDD) for 6 weeks as described by Nakano et al. (Hepatology (26): 670-678, 1997). Rats fed a CMDD exhibited a time-dependent increase in liver triglyceride (TG) content from ˜10 to 250 mg TG/g liver after 5-6 weeks (FIG. 8A). This accumulation was reversible, as returning the animal back to a regular diet caused the hepatic TG content to return to normal (FIG. 8B).

CMDD fed rats were returned to a regular diet for 3 or 7 days before harvesting the livers for transplantation. The donor livers were removed and stored as follows. After laparotomy, the bile duct of the liver was cannulated with a short polyethylene tube. Veins emptying into the portal vein and the hepatic artery were subsequently ligated and divided, and the portal vein was divided at the level of the inferior mesenteric vein. To prepare the portal vein cuff, a short polyethylene tube was slipped over the vein and the vein everted over the tube. The infrahepatic vena cava and suprehepatic vena cava, including part of the diaphragm, were then transected. The liver is flushed with hetastarch-free UW solution and stored in a reservoir containing the same for 6 hours at 0° C.

The donors were stored in a hetastarch-free UW preservation solution for 6 hours at 4° C., and then transplanted into a recipient rat as follows. The recipient animal was prepared by cannulating the bile duct, clamping the portal vein, and tying shut the other vessels. The liver was removed and discarded. The donor liver is placed orthotopically, the suprahepatic vena cava anastomosed, and the cuffed portal vein was inserted into the recipient's portal vein. Blood is then allowed to flow into the donor liver, and the infrahepatic vena cava is anastomosed. The bile duct is reconnected and wrapped around the omentum. The abdominal incision is then closed. This protocol mimics the clinical situation which typically requires that the liver be preserved in the UW solution for several hours while it is being transported from the donor to the recipient site.

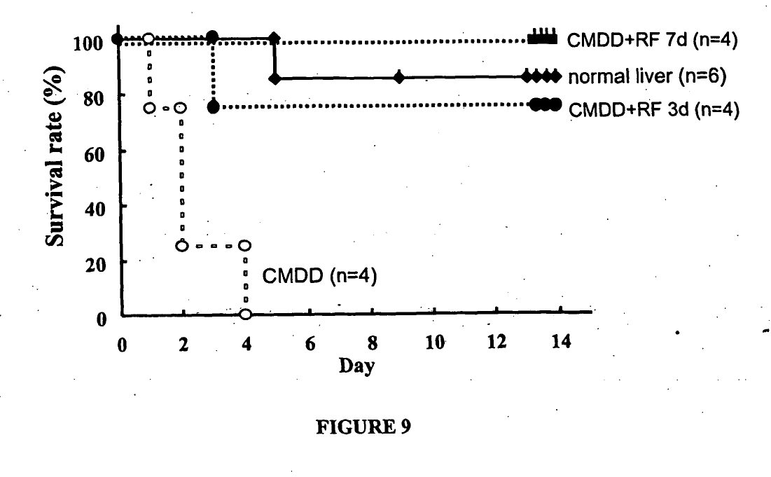

As is shown in FIG. 9, the control animals receiving untreated fatty livers did not survive after 4 days. In contrast, recipients of defatted livers showed a complete recovery of survival rate, with no statistically significant difference in survival when compared to recipients receiving control (nonfatty) livers.

Metabolic Preconditioning of Steatotic Perfused Livers to Reduce Their Lipid Content

Based on our cell culture data, we tested the effect of warm perfusion with buffer containing no insulin and high glucagon (10 ng/mL) on the triglyceride content of steatotic livers. Donor livers were prepared for transplantation and then perfused at 37° C. as follows. Steatotic livers were obtained by feeding rats a CMDD for 6-7 weeks. The buffer also contained 3% bovine serum albumin in order to prevent tissue swelling. Perfusions were carried out at a flow rate of 4 mL/min/g liver, 37° C., and using 95% O₂/5% CO₂ for 1-3 hours in a recirculating mode. The perfusate solution consists of Minimal Essential Medium supplemented with hydroxyethyl starch (6% w/v), amino acids, glucagon, hydrocortisone, and anti-oxidants.

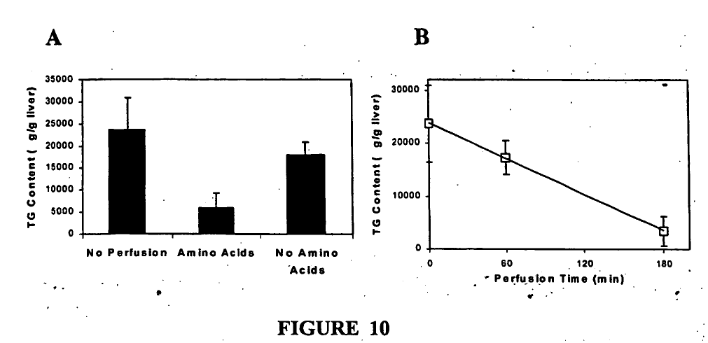

The triglyceride content of livers was measured after the perfusion and compared to that of unperfused livers from rat littermates. Initially, we compared buffer vs. amino acid-containing medium, and found a significantly increased rate of triglyceride clearance in the presence of amino acids (FIG. 10A). Using amino acid-supplemented medium, we investigated the kinetics of clearance during the first 3 hours of perfusion, and found a linear relationship (FIG. 10B). After 3 hours, warm perfusion reduced the triglyceride content of fatty livers by 85%. These data demonstrate that warm perfusion can be used to reduce the hepatic lipid storage of fatty livers.

In addition, as is shown in FIG. 10B, the TG content decreased as a function of time and the defatting process was largely complete after 3 hours. It is likely that there are two major mechanisms of action of the defatting regimen. First, the catabolic hormones glucagon and hydrocortisone, which are in the perfusate, favor the oxidation of lipids, more specifically fatty acids. Second, the amino acids in the perfusate provide the building blocks required for the synthesis of apolipoproteins, which are then incorporated into the larger lipoproteins. These lipoproteins export TG and other lipids (e.g. cholesterol) outside of the cell.

It is interesting to note that, based solely on typical measured oxygen uptake rates of perfused livers, one would predict a maximum possible rate of lipid oxidation about one order of magnitude less than observed in FIGS. 8A and 8B, suggesting that other pathways of defatting (e.g. export of triglycerides in the form of lipoproteins) are probably very important in this process. In addition, using the triglyceride clearance equation (equation 1) fitted to cell culture data earlier would predict a decrease to 82% of the original lipid load after 3 hours of treatment with low insulin medium (as compared to the 85% measured), suggesting that our steatotic hepatocyte culture model closely reflects the behavior of fatty livers, and thus can be used to rapidly screen for more effective defatting regimens.

To summarize, fatty livers are very sensitive to ischemia-reperfusion and cold preservation-related injuries, which makes them unacceptable for liver transplantation. We hypothesized that removal of the excess fat storage from fatty livers can restore their ability to undergo liver transplantation. We obtained fatty livers from rats fed a CMDD for 6 wk, stored them in cold hetastarch-free UW solution for 6 hours, and transplanted them into normal recipient rats. While recipient rats had a 90% rate of survival after transplantation of control normal lean livers, they all died when receiving CMDD rat livers. If CMDD rats were returned to a normal diet for 3 or 7 days prior to donating livers, effectively reducing the fat content of the livers by 33% and 85%, respectively, the recipients survived at rates similar to the controls. Furthermore, we found that it is possible to eliminate excess fat storage from fatty livers by short-term perfusion of fatty livers ex vivo. These results support the notion that liver perfusion could be used to recondition fatty livers and make them suitable for transplantation.

Heat Shock Preconditioning of Steatotic Livers Increases Ischemic-Reperfusion Injury

We next investigated the effect of heat shock preconditioning (HPc) on recipient survival in the rat fatty liver transplantation model. Fatty liver donor rats were exposed to brief whole body hyperthermia (10 min at 42.5° C.) and allowed to recover.

Heat Shock Increases the Expression of Heat Shock Proteins

We first characterized the dynamics of induction of HSPs (see FIGS. 11A-11C). We compared HSP72 levels in livers from CMDD-fed and normal lean rats up to 72 hours after HPc. In steatotic livers, HSP72 levels measured by enzyme-linked immunosorbent assay (ELISA) increased until 12 hours after HPc and were highest between 12 and 24 hours after HPc (FIG. 11A). Interestingly, this induction occurred faster than in normal lean controls, in which HSP72 levels peaked at 48 hours. Using western blot analysis, we then analyzed HSP72, heme oxygenase-1 (HO-1) and HSP90 contents in livers from CMDD-fed rats up to 240 hours after HPc (FIGS. 11B and 11C). We detected HSP72 and HO-1, both inducible HSPs, as early as 3 hours after HPc. HSP72 levels were highest 6-24 hours after HPc, consistent with our ELISA data, while HO-1 was highest 12-48 hours after HPc. HSP90, a constitutive HSP, was detectable in controls and did not change until 12 hours after HPc, after which it increased to stabilize 24-48 hours after HPc, and decreased afterwards. Overall, we found that HPc induced heat shock proteins (HSP72, HSP90, and heme oxygenase-1) in donor livers, with levels peaking 12 to 48 hours after HPc. For subsequent transplantation studies, we chose to harvest donor livers 24 hours after HPc because all HSPs were highly expressed at that time point.

Effects of HPc on Liver Injury and Serum Cytokines after Transplantation.

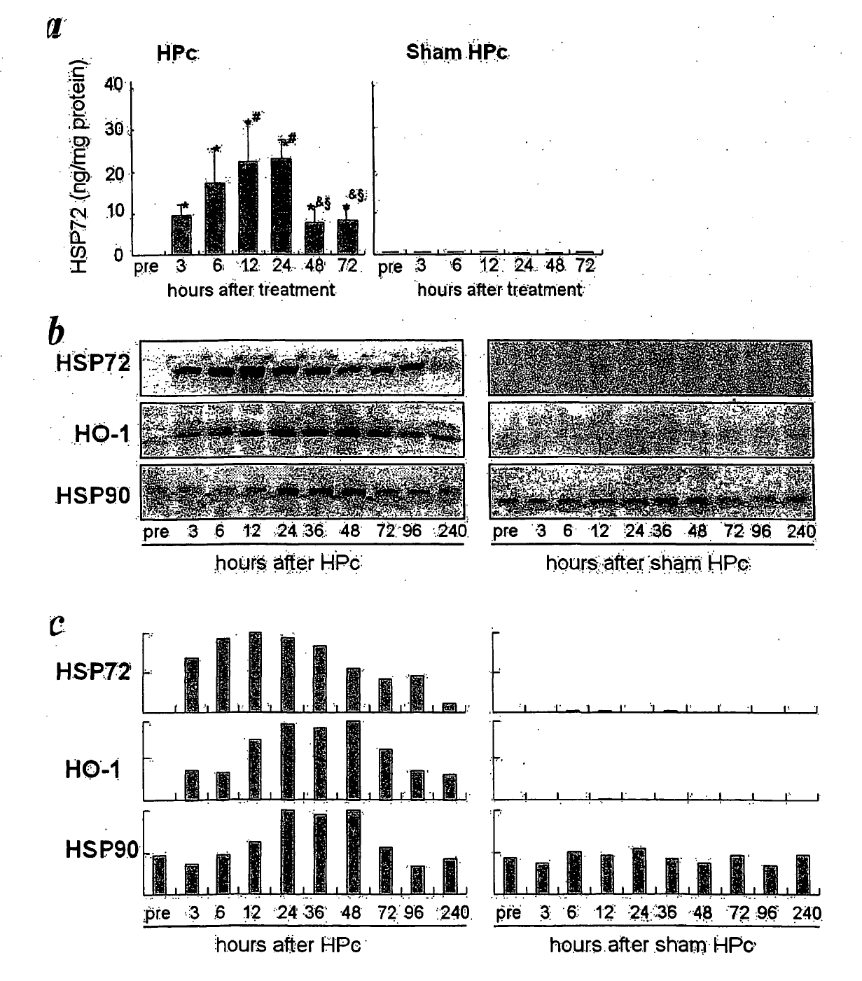

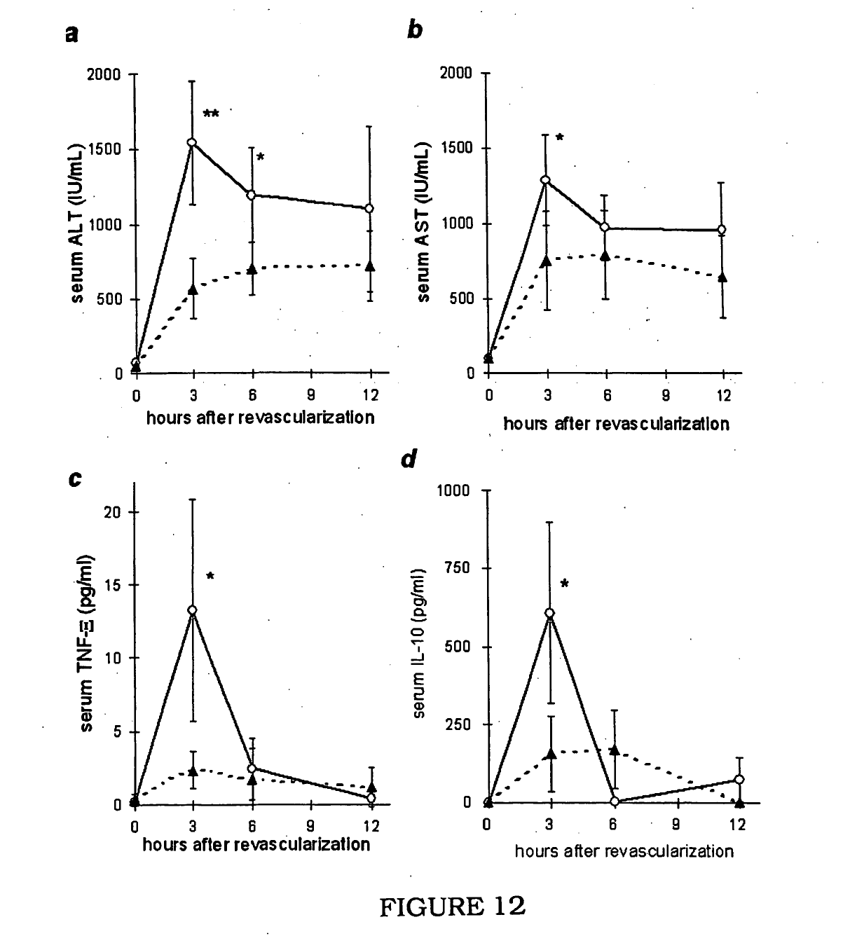

Prior to transplantation, we stored donor livers in hetastarch-free University of Wisconsin (UW) solution for 10 hours at 4° C. Following transplantation in recipients, we measured serum levels of alanine aminotransferase (ALT) and aspartate transaminase (AST) (both of which reflect hepatocellular injury) as well as serum levels of tumor necrosis factor alpha (TNF-α) and interleukin (IL-) 10 (used as indexes of systemic inflammation) (see FIGS. 12A-12D). AST and ALT levels peaked 3 hours after transplantation of sham-treated livers and remained elevated until at least the 12 hour time point. Transplantation of HPc livers moderated the initial increase in ALT and AST, although the values were not significantly different from controls after the 12 hour time point. In controls receiving sham-treated livers, TNF-α and IL-10 levels dramatically increased 3 hours after transplantation. In contrast, HPc treatment almost completely abrogated the elevation in cytokine levels, suggesting inhibition of the inflammatory response.

Effects of HPc on Histology after Transplantation

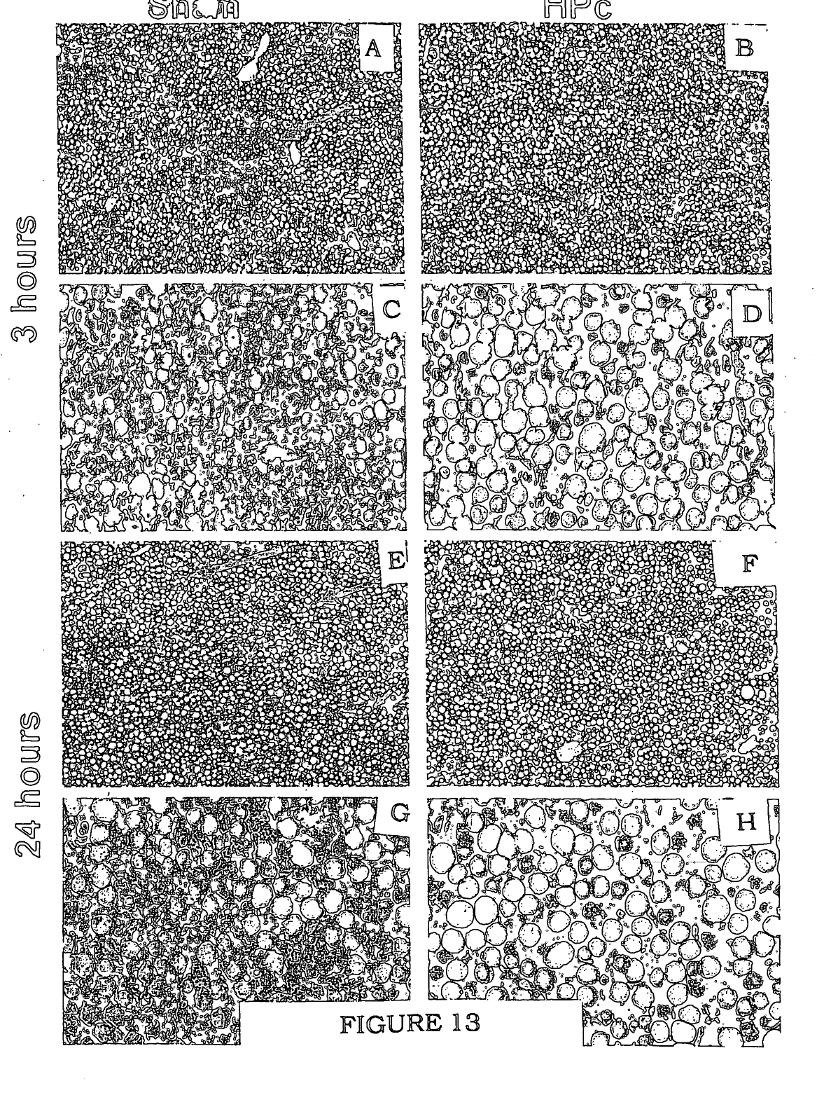

Histologic examination of transplanted steatotic livers demonstrated severe congestion and confluent hemorrhagic change 3 and 24 hours after transplantation (see FIGS. 13A, 13C, 13E, and 13G). Fulminant hepatocellular necrosis was also apparent 24 hours after transplantation (FIGS. 13E and 13G). In contrast, HPc livers displayed greatly reduced hemorrhagic injury and necrosis arising in a sparse pattern (FIGS. 13B, 13D, 13F, and 13H). It is noteworthy that, in all cases, hepatic steatosis was evident and that there were no qualitative histologic differences between HPc and sham-treated livers in that respect. Liver histology of the two groups prior to transplantation also showed no difference in the severity of steatosis.

Donor livers were subsequently harvested 24 hours after HPc, placed in cold storage for 10 hours, and transplanted into normal rats. At 3 hours post-transplantation, HPc reduced serum liver enzymes in the recipients, and almost completely suppressed the release of TNF-α and IL-10. Histological evaluation 3 and 24 hours after transplantation show that HPc significantly reduced hepatic inflammation and hepatocellular necrosis without affecting the steatotic appearance of hepatocytes.

Heat Shock Preconditioning Increases Transplantation Survival

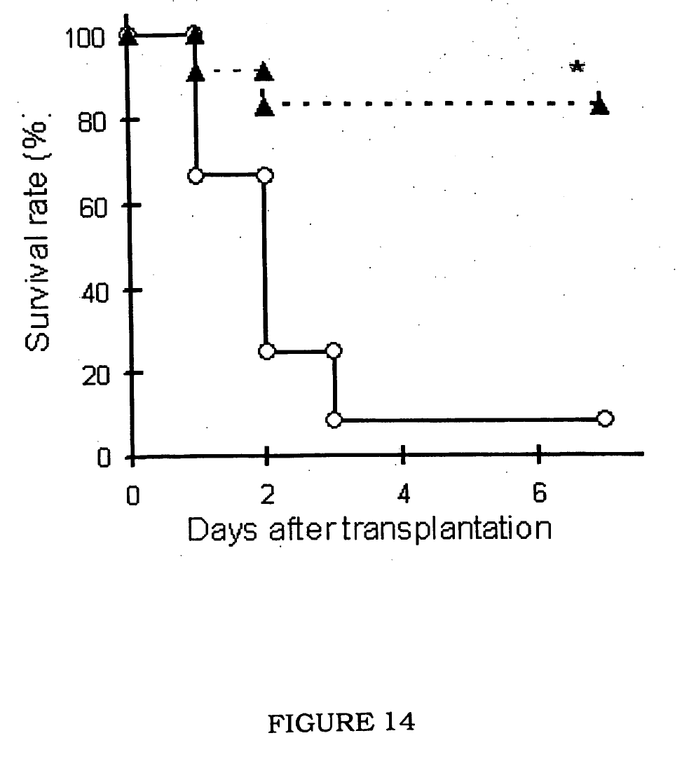

We monitored the effect of HPc of donor fatty livers on the survival of recipient rats for up to 1 week (FIG. 14). 11 out of 12 recipients receiving sham HPc livers died of primary graft dysfunction within 3 days following transplantation. In contrast, HPc resulted in recipient survival exceeding 80% (10 out of 12). This survival rate was comparable to that seen with normal lean livers (86% or 6 out of 7) that were transplanted using the same protocol in the absence of heat shock treatment. Thus, HPc induced tolerance of fatty livers to cold I/R injury associated with transplantation.

Effects of Recovery Time and HSP Levels after HPc on Survival Rate of Recipient Rats after Transplantation

We next determined the sensitivity of recipient survival rate to the recovery time period after HPc. In these experiments, we harvested donor livers between 3 and 72 hours after HPc or sham preconditioning, and stored the livers for 10 hours at 4° C. prior to transplantation. The data indicate that the protective effects of HPc were present as early as 3 hours after HPc, and reached their maximal effect at 6 hours after HPc (Table 3, which shows the effect of recovery time after HPc of donor livers on the survival rate of recipients).

TABLE 3

| | | | | | Statistical | |
|---|---|---|---|---|---|---|
| Recovery | | | | | significance | |
| period | Survival rate on day 7 | | | | | vs. 24 hr |
| (hr) | HPc | | Sham | | vs. sham | recovery |
| 3 | 33.3% | (3/9) | 0.0% | (0/6) | P < 0.05 | P < 0.05 |
| 6 | 77.7% | (7/9) | 5.0% | (0/6) | P < 0.01 | N.S. |
| 24* | 83.3% | (10/12) | 8.3% | (1/12) | P < 0.005 | — |
| 48 | 83.3% | (5/6) | 16.7% | (1/6) | P < 0.05 | N.S. |
| 72 | 30.0% | (3/10) | 10.0% | (1/10) | N.S. | P < 0.01 |

*time course shown in FIG. 14

There was no significant difference in the survival rates among the 6, 24, and 48 hours groups. However, the protective effect of HPc had disappeared 72 hours after HPc, therefore suggesting that the maximal protective effects of HPc occurs in a window of time between 6 to 48 hours after HPc.

One week after transplantation, non-heat shocked control transplants exhibited a survival rate <10%, while heat shocked fatty liver recipients survived >80% of the time. Evaluating the survival of recipients receiving fatty livers at different times following HPc revealed that the protective effect of HPc was significant when donors were transplanted 3-48 hours after HPc, with the maximal effect seen 6-48 hours after HPc. Accordingly, HPc is a promising avenue to salvage rejected donor fatty livers and enhance the survival rate of fatty liver recipients. This technique could significantly increase the annual donor pool supply.

Induction of HSPs after HPc and GdCl₃

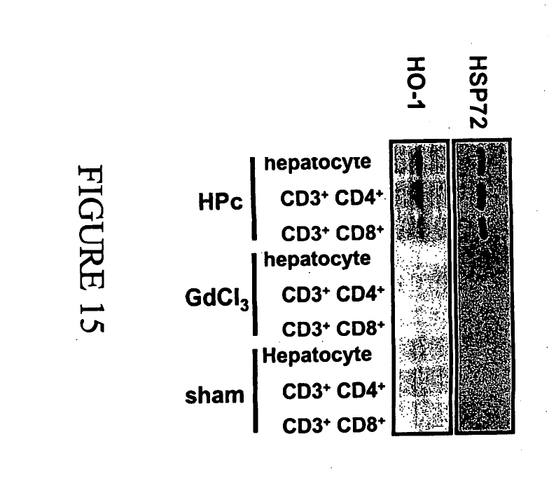

In order to detect the expression of such HSPs in T cells, western blot analysis was performed in CD4⁺ or CD8⁺ T cells and compared to that of hepatocytes in Sham, HPc, and GdCl₃ groups at the time of harvesting (FIG. 15). In the HPc group, HSP72 and HO-1 were not only expressed in hepatocytes, but also in CD4⁺ and CD8⁺ T cells. On the other hand, neither HSP72 nor HO-1 was detected in any of these cells in both the Sham or GdCl₃-treated groups.

Effects of HPc and GdCl₃ on Liver Injury after Transplantation

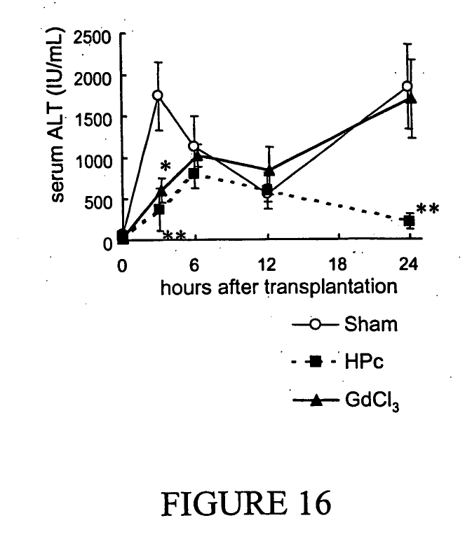

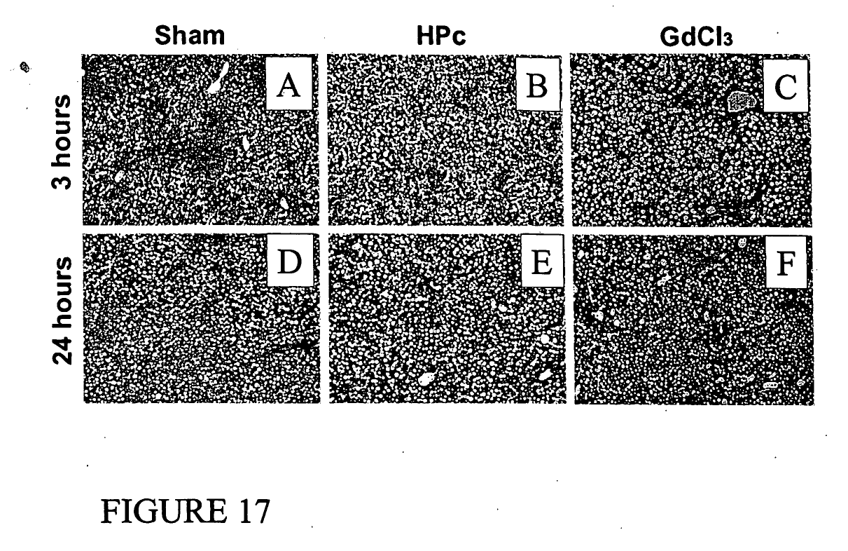

Liver injury after liver transplantation was determined by assessing the levels of serum ALT (FIG. 16) and by histological analysis (FIGS. 17A-17F). Serum ALT level of the Sham group that underwent liver transplantation after 12 hours cold preservation demonstrated a biphasic pattern of liver injury that peaked at 3 hours and 24 hours, representing early acute and subacute damage, respectively. In comparison, the GdCl₃-treated group of rats demonstrated that the ALT levels 3 hours after transplantation were significantly lower than that of Sham group. In addition, the HPc-treated group exhibited ALT levels that were significantly lower than that of the Sham group at 3 hours and 24 hours after transplantation. Consistent with serum ALT activities, histologic examination of transplanted Sham group livers demonstrated severe congestion and confluent hemorrhagic change 3 and 24 hours after transplantation (FIGS. 17A and 17D). Fulminant hepatocellular necrosis was also apparent 24 hours after transplantation (FIG. 17D). In contrast, GdCl₃ livers displayed reduced hemorrhagic injury 3 hours after transplantation, compared to the Sham group livers (FIGS. 17C and 17F). Moreover, HPc livers displayed greatly reduced hemorrhagic injury and necrosis both of which arose in a sparse pattern (FIGS. 17B and 17E). Liver histology of the three groups prior to transplantation showed no difference in the severity of steatosis.

Effects of HPc and GdCl₃ on the Serum Cytokine Levels after Transplantation

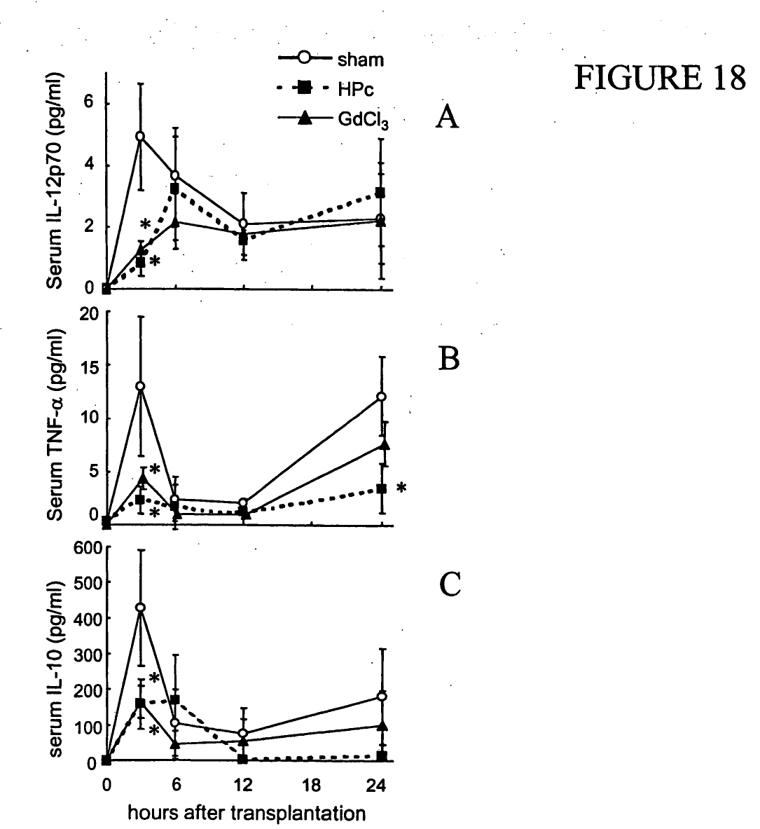

Heat shock preconditioning is known to suppress the production of cytokines, such as TNF-α, and to reduce the accumulation of neutrophil after I/R injury in the liver. We measured serum levels of IL-12p70, TNF-α, and IL-10, which were produced mainly by Kupffer cells (FIGS. 18A-18C). IL-12, TNF-α, and IL-10 peaked 3 hours after transplantation of Sham group livers and TNF-α levels also demonstrated a biphasic pattern peaking at 3 hours and 24 hours. Transplantation of HPc and GdCl₃ livers moderated the initial increase in IL-12, TNF-α, and IL-10 significantly. Moreover, TNF-α levels in the HPc group 24 hours after transplantation were significantly suppressed relative to that of the control group. In contrast, such levels in the livers of the GdCl₃-treated group were not significantly different from that of liver controls after the 24 hours time point. Serum IL-4 or IFN-γ was not detected in any group at any stage after transplantation.

Effects of HPc and GdCl₃ on the Neutrophil Accumulation in the Liver after Transplantation

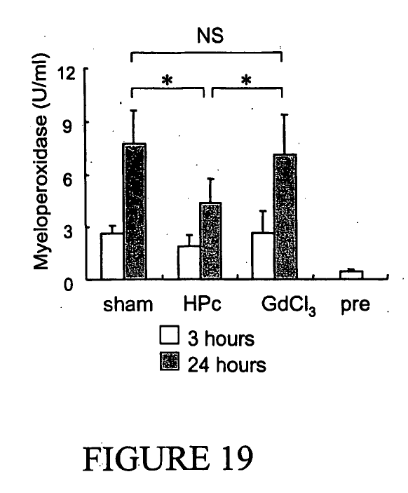

Next, we determined the neutrophil accumulation to measure MPO content of the liver tissues (FIG. 19). In the Sham group, MPO contents were increased 3 times and 18 times at 3 hours and 24 hours following reperfusion, respectively, compared with levels prior to transplantation. Three hours after transplantation, MPO levels of the Sham, GdCl₃-treated, and HPc groups were almost similar with no significant difference. On the other hand, there was a significant difference in MPO levels in the Sham group and HPc group 24 hours after the transplantation although there is no difference between the Sham group and the GdCl₃-treated group.

Effects of HPc and GdCl₃ on Survival Rate of Recipient Rats after Transplantation

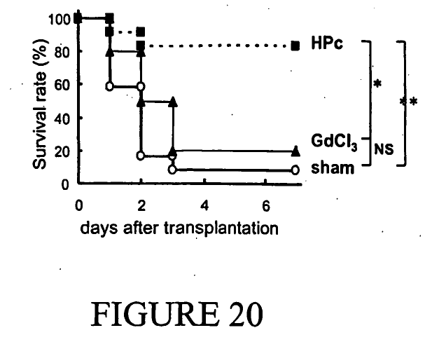

Donor fatty livers were HPc or GdCl₃ treated and the effect of such treatment on the survival of recipient rats was monitored for up to 1 week after transplantation. Survival curves of rats that underwent liver transplants are shown in FIG. 20. In transplantation cases with Sham group livers, 11 out of 12 recipients died of primary graft malfunction 3 days following transplantation. Despite improvements in serum ALT levels and structural amelioration (as shown by histological analysis) 3 hours after transplantation, there was no significant difference between the recipient survival rate of Sham and GdCl₃-treated groups. In contrast, the recipient survival rate of HPc group livers was dramatically improved.

Liver injury after liver transplantation with cold preservation is caused mainly by I/R injury. We next studied if HPc had an effect on the levels of monokines released from Kupffer cells and if such levels reduced neutrophil accumulation in the liver, in turn suppressing liver injury. Pretreatment with GdCl₃ suppressed liver injury and TNF-α, IL-10, and IL-12 release 3 hours after transplantation. However, GdCl₃ did not suppress liver injury or neutrophil accumulation 24 hours after transplantation. Moreover, GdCl₃ did not improve the recipient survival rate. These results indicate that while suppression of Kupffer cells improved liver injury in the acute phase (3 hours after transplantation), the same was not true for the subacute phase (24 hours after the transplantation). Furthermore, liver injury during the subacute phase was more critical in graft survival rate.

Effects of HPc and GdCl₃ on Liver T Cells after Transplantation

Our findings that HPc, in contrast to GdCl₃ (an agent that suppresses Kupffer cell activity) improved both acute and subacute phase liver injury suggest that other cells such as T cells may play important roles in liver injury and HPc protection in the liver. We next examined the effect of HPc on the relative numbers of CD4⁺ T cells and CD8⁺ T cells in the liver after transplantation by flow cytometry (Table 4, which shows the effects of HPc and GdCl₃ on the relative number of T cells after transplantation).

TABLE 4

| | | 3 hr | | | 24 hr | | |
|---|---|---|---|---|---|---|---|
| | naive | Sham | HPc | GdCl₃ | Sham | HPc | GdCl₃ |
| CD3⁺CD4⁺ | 5.2 ± 1.4 | 7.5 ± 1.8 | 4.7 ± 2.5 | 8.4 ± 2.7 | 8.9 ± 2.7 | 4.8 ± 1.3* | 7.2 ± 2.2 |
| CD3⁺CD8⁺ | 3.1 ± 1.5 | 4.8 ± 1.2 | 3.2 ± 1.0 | 4.2 ± 1.2 | 5.9 ± 1.5 | 3.2 ± 0.9* | 5.6 ± 2.3 |

Data are expressed as mean ± SD (×10⁵).
*P < .05 versus the Sham group.

There was no difference in the numbers of CD3⁺ CD8⁺ cells in the liver between the Sham, GdCl₃, and HPc groups at any stage following transplantation. On the other hand, CD3⁺ CD4⁺ cells appeared to decrease in numbers in the livers of the HPc group compared to that of the control group 24 hours after transplantation.

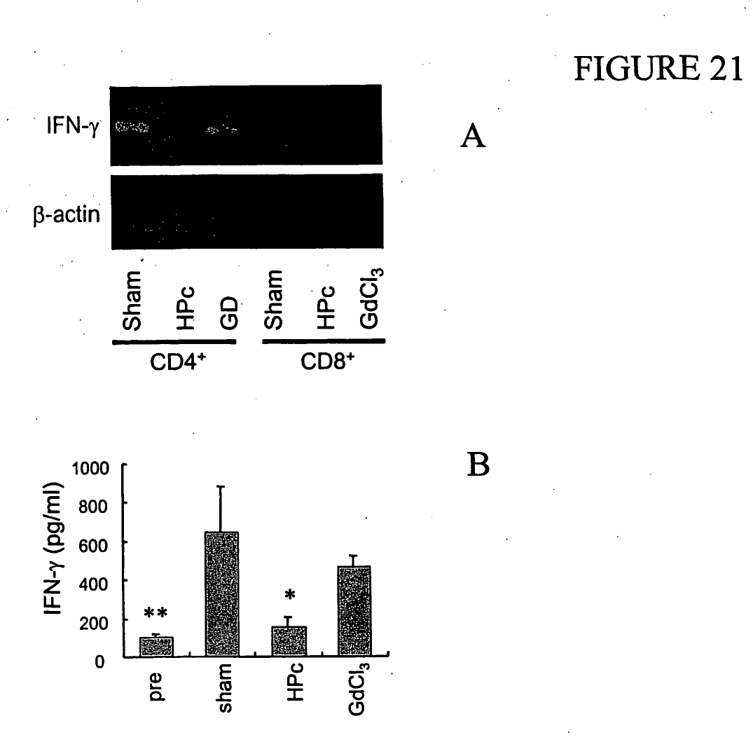

To determine the functional difference in liver T cells between the Sham, GdCl₃, and HPc groups, we purified T cells bearing CD3⁺ CD4⁺ and CD3⁺ CD8⁺ from lymphocytes of liver transplanted 24 hours and examined the expression of mRNA specific for IFN-γ and IL-4 by means of cytokine RT-PCR. As shown in FIG. 21A, the expression level of IFN-γ mRNA in isolated CD3⁺ CD4⁺ cells from C and GdCl₃ groups was much higher than that from HPc group, whereas that expression level in isolated CD3⁺ CD8⁺ cells remained low in all three groups. The expression of IL-4 mRNA was not detected in any group.

We next examined cytokine production by liver T cells from livers 24 hours after transplantation in response to immobilized anti-CD3 mAb. As shown in FIG. 21B, IFN-γ production by liver CD4⁺ T cells from the livers of the HPc group was significantly decreased compared with that by CD4⁺ T cells from the livers of the Sham and GdCl₃ groups.

Effects of CyA Pretreatment of Donor Rat on Liver Injury after Transplantation

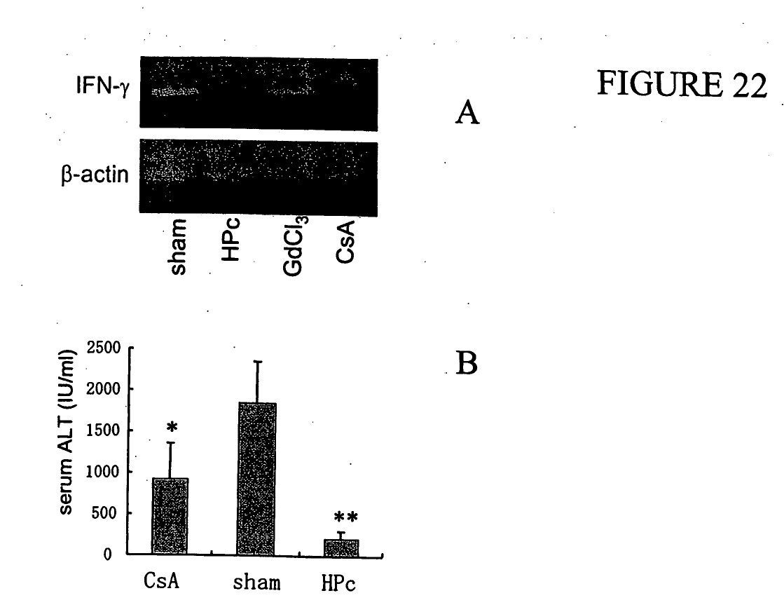

We found that the levels of CD4⁺ T cells were suppressed in HPc donor livers after transplantation. To determine whether the suppression of liver T cells was responsible for the protective effect of HPc on liver injury after transplantation, rats were injected i.v. with CyA, a potent T cell-deactivating-agent, 6 hours before the harvesting of donor livers. As shown in FIG. 22A, the administration of CyA diminished the expression of IFN-γ mRNA as much as HPc 24 hours after transplantation. We assessed liver injury based on the serum levels of ALT 24 hours after the liver transplantation. As shown in FIG. 22B, serum ALT levels in CyA-treated liver was significantly lower than that in the Sham group, but significantly higher than that of the HPc group. Taken together, these results suggested that, suppression of liver T cells is partly responsible for the protection of HPc on liver injury after transplantation.

The above experiments were performed using the following methods and materials.

### Methods

Induction of Hepatic Steatosis in Donor Rats.

Several fatty liver rat models are available for experimental purposes. In addition to genetically obese animals (which have steatotic livers), the accumulation of lipids in animal models may be induced, for example, from alcohol administration, lipotrope diets, and choline and methionine deficient diets. Regarding the choline and methionine deficiency model, which is used herein, choline and methionine are essential precursors for the synthesis of very low density lipoproteins. The lack of choline and methionine in the diet therefore blocks the export of triglycerides from hepatocytes, resulting in fat accumulation in the liver. Within a few weeks of such a diet, rats develop a severe-grade hepatic steatosis, predominantly macrovesicular, without any evidence of inflammation and/or fibrosis. Triglycerides are the main component of the accumulated fatty droplets with an increased molar percentage of palmitic and oleic acids. Because of the pathological and biochemical similarities of this model relative to fatty livers in humans, particularly in cases of rich carbohydrate diets, we have chosen the choline- and methionine-deficient model to study ischemia-reperfusion injury in steatosis liver. We also used a syngeneic rat model of liver transplantation, which includes a 6 to 12 hour period of cold preservation in UW solution. This experimental protocol was designed based on a typical liver transplantation procedure in a clinical setting requiring that the donor liver be stored and transported in ice-cold UW solution for several hours. An inbred strain of rats was used to eliminate the effects of allogeneic rejection. In sum, such an experimental model was the most suitable model to study the I/R injury in the steatotic liver transplantation.

All procedures with animals were approved by the Subcommittee on Research Animal Care, Massachusetts General Hospital and in accordance with National Research Council guidelines. Male Lewis rats (Charles River, Wilmington, Mass.) weighing 280 to 320 g were housed in a 12 hours day-light cycle and allowed free access to food and water. To induce fatty liver, the rats were CMDD-fed (Test Diet, Richmond, Ind.) for 40 to 44 days.

Experimental Groups and Treatments of Donor Rats

Donor animals were divided into three groups; heat shock preconditioning (HPc) group, sham HPc (Sham) group, and gadolinium chloride (GdCl₃) group. In the HPc group, rats were anesthetized and placed in a waterproof bag that was then immersed in a 43° C. water bath to elevate the core body temperature (measured via a rectal digital thermometer) to 42-42.5° C. Animals were maintained at that temperature for 10 min and then removed from the warm bath. Animals then received 10 ml/kg intraperitoneal saline injection and were allowed to recover with free access to food and water. Animals in the Sham and GdCl₃ groups underwent the same procedures except that they were immersed in a 37° C. bath (sham HPc). In the GdCl₃ group, Kupffer cell deactivation was achieved by two 20 mg/kg GdCl₃ (Sigma, St. Louis, Mo.) intravenous injection 48 and 24 hours before donor liver harvesting. In the Sham and HPc groups, sterile nonpyrogenic saline was used as instead of GdCl₃ solution. Donor livers were harvested 24 hours after HPc or sham HPc. In some experiments, rats were pretreated with 5 mg/kg i.m. Cyclosporine A (CyA; Sigma) 6 hours before donor liver harvesting.

Donor Liver Removal, Preservation, and Transplantation

Isogenic orthotopic liver transplantation was performed as described by Kamada and Calne (Kamada et al., Transplantation 28:47-50, 1979). Donor livers were harvested 3, 6, 24, 48, or 72 hours after HPc. Briefly, the bile duct was cannulated with a short intraluminal polyethylene stent. Veins emptying into the portal vein and the hepatic artery were ligated and divided, and the portal vein divided at the level of the inferior mesenteric vein. The infrahepatic and suprahepatic vena cava, including part of the diaphragm, were transected. The liver was flushed with 10 mL cold saline containing 50 U heparin and 5 ml hetastarch-free University of Wisconsin solution (Sumimoto et al., Transplantation 48:1-5, 1989) and subsequently stored for 6-12 hours at 4° C. After cold storage, orthotopic liver transplantation was performed without hepatic artery reconstruction. The donor liver was flushed with 6 ml cold Ringer's solution, the suprahepatic vena cava anastomosed with a 7-0 nylon running suture, and the portal vein anastomosed using the cuff technique. Blood was allowed to flow into the donor liver, and the infrahepatic vena cava anastomosed using the cuff technique. After revascularization of the graft, the rat was given 8 ml/kg Ringer's solution and 2 mL/kg 7% w/v NaHCO₃ intravenously, and intramuscular injections of 80 mg/kg penicillin and 100 mg/kg streptomycin. The bile duct was connected and wrapped around the omentum. Anhepatic time ranged from 14 to 16 minutes. For survival studies, the animals were returned to standard housing facilities and monitored for up to one week. In the case of animals used for biochemical and histological studies, animals were sacrificed 3 hours, 6 hours, 12 hours, or 24 hours after transplantation. Blood samples were collected from the hepatic vein draining the left lateral lobe, as previously described (Kamada et al, supra).

Liver Triglyceride Content

Liver tissue was sonicated in 20 volumes of 0.25 M sucrose, 50 mM Tris-HCl, 1 mM EDTA for 1 min at 4° C. Triglyceride concentration in the homogenate was measured using a commercial kit (Sigma Chemical, St. Louis, Mo.).

Western Blot Analysis for HSP72, HSP90, and HO-1

Donor rats were sacrificed up to 240 hours after HPc. Liver tissue was homogenized in 4 volumes of 0.25 M sucrose for 30 seconds at 4° C. Liver proteins were separated by sodium dodecylsulfate-polyacrylamide gel electrophoresis and transferred to polyvinylidene difluoride membranes (Sigma Chemical). Antibodies to detect HO-1 (1:2,000), HSP 72 (1:1,000), and HSP90 (1:1,000) were mouse monoclonals from Stressgen (Victoria, British Columbia, Canada). The secondary antibody was a peroxidase-conjugated goat-anti mouse IgG (Stressgen) diluted 1:10,000. Protein signals were visualized by chemiluminescence (Pierce, Rockford, Ill.) and recorded on a GS282 Scanner (BioRad, Hercules, Calif.).

ELISA for HSP72, TNF-α and IL-10

HSP72 levels in the liver homogenates and TNF-α and IL-10 levels in serum were determined by ELISA. HSP72 was analyzed using a commercial kit (Stressgen). TNF-α and IL-10 were analyzed using R&D Systems (Minneapolis, Minn.) monoclonal antibodies according to the manufacturer's instructions.

Biological Assays

To assess liver injury, alanine aminotransferase (ALT) was measured in serum samples using a commercially available kit (Sigma). TNF-α, IL-10, IL-4, IL-12, and IFN-γ levels in serum and cell culture supernatant were determined by Enzyme-linked immunosorbent assay (ELISA). ELISA for IL-12p70 was performed using Biosource (Camarillo, Calif.) kit. ELISAs for others were performed using R&D systems (Minneapolis, Nebr.) mAbs according to the manufacture's instructions.

Blood Chemistry

Blood samples were collected from the hepatic vein draining the left lateral lobe (Yoshioka et al., Hepatology 27:1349-1353, 1998). To assess the extent of liver injury, ALT and AST were measured in serum using a commercial kit (Sigma Chemical).

Preparation of Liver Lymphocytes and Enrichment of CD4⁺ and CD8⁺ T Cells

Three hours or 24 hours after liver transplantation, transplanted liver were perfused with sterile PBS through the portal vein to wash out all remaining peripheral blood and then meshed with stainless steel mesh. After the coarse pieces were removed by centrifugation at 50 g for 1 min, the cell suspensions were again centrifuged, resuspended in 8 mL of 45% Percoll (Sigma), and layered on 5 mL of 67.5% Percoll. The gradients were centrifuged at 600 g at 20° C. for 20 min. Lymphocytes at the interface were harvested and washed twice with PBS. CD4⁺ or CD8⁺ T cells were purified by Rat T cell CD4 or CD8 column kit (R&D systems, Minneapolis, Nebr.) from the harvested liver lymphocytes. The purity of sorted cells was more than 95%.

Flow Cytometry Analysis

For 3-color analysis, liver lymphocytes were incubated with saturating amounts of phycoerythrin-conjugated anti-rat CD3α mAb (Pharmingen, San Diego, Calif.) and fluorescein isothiocyanate-conjugated anti-rat CD4 mAb (Pharmingen) for 30 min. Cells were analyzed with a FACSCalibur flow cytometer (Becton Dickinson, San Jose, Calif.). We carefully gated cells by forward and side light scattering for the liver lymphocytes. The data were analyzed using CyQuest software (Becton Dickinson).

Reverse Transcription-Polymerase Chane Reaction (RT-PCR)

Total RNA was extracted by the acid guanidium-phenol-chloroform method from Isolated CD4⁺ and CD8⁺ T cells. Complementary DNA (cDNA) synthesis and polymerase chain reaction (PCR) were performed using a complementary DNA cycle kit (Invitrogen Corp., San Diego, Calif.). The PCR was performed on a PCR thermal cycler (Applied Biosystems, Foster city, CA). PCR cycles were run for 30 sec at 94° C., 30 sec 54° C., and 30 sec at 72° C. with 30 cycles. The specific primers were as follows: IL-4 sense, 5′-GAA CCA GGT CAC AGA AAA AGG-3′ (SEQ ID NO: 1); IL-4 antisense, 5′-CTG CAA GTA TTT CCC TCG TAG G-3′ (SEQ ID NO: 2); IFN-γ sense, 5′-CAC GAA AAT ACT TGA GAG CC-3′ (SEQ ID NO: 3); IFN-γantisense, 5′-TCT CTA CCC CAG AAT CAG CACC-3′ (SEQ ID NO: 4). The PCR product was subjected to electrophoresis on a 1.5% agarose gel (Life Technologies).

Lymphocyte-Associated Cytokine Assays

Liver lymphocytes were obtained by the same method previously described. Tissue culture 96-well plates were incubated overnight at 4° C. with 50 mg/mL anti-CD3ε mAb (Pharmingen). The plates were then washed thoroughly. The harvested lymphocytes (5×10⁵/well) were incubated in the anti-CD3ε mAb-coated plates for 48 hours. IFN-γ and IL-4 levels in the culture supernatants were determined by ELISA.

Assessment of Neutrophil Infiltration

The presence of myeloperoxidase (MPO), enzyme specific for neutrophil (and some macrophages), was used as an index of intrahepatic neutrophil accumulation. Briefly, the frozen tissue was thawed weighed, and placed in 4 mL iced 0.5% hexadecyltrimethylammonium bromide and 50 mM potassium phosphate buffer solution with the pH adjust to 5. Each sample was then homogenized for 30 sec and centrifuged at 12000 g for 20 min at 4° C. Supernatants were then mixed with hydrogen peroxide-sodium acetate and tetramethyl-benzidine solutions. The change in absorbance was measured by spectrophotometry at 450 nm. One unit of MPO activity was defined as the quantity of enzyme degrading 1 μM peroxide per minute at 25° C. per gram of tissue.

Histology

Animals were sacrificed before or 24 hours after HPc, and 3 or 24 hours after transplantation. Livers were fixed in 10% buffered formalin, embedded in paraffin, thin-sectioned, and stained with hematoxylin and eosin for transmission brightfield microscopic examination. Steatosis was graded semiquantitatively as described elsewhere (Koneru et al., Transplantation 73:325-330, 2002 and Adam et al., Transplant Proc. 23:1538-1540, 1991).

Statistical Analysis

Data are expressed as means±SD. Difference among groups were determined using ANOVA and post hoc Tukey's test, except for survival studies, where the generalized Wilcoxon test was used. Differences were deemed to be significant when P<0.05.

### Other Embodiments

All publications mentioned in this specification are hereby incorporated by reference to the same extent as if each independent publication or patent application was specifically and individually indicated to be incorporated by reference.

What is claimed is:

1. A method for preparing a donor cell, tissue, or organ for transplantation into a recipient, said method comprising reducing intracellular lipid storage material of said cell, tissue, or organ.

2. The method of claim 1, wherein a donor cell is prepared.

3. The method of claim 1, wherein a donor tissue is prepared.

4. The method of claim 1, wherein a donor organ is prepared.

5. The method of claim 1, wherein said cell is a liver cell, said tissue is a liver tissue, or said organ is a liver.

6. The method of claim 1, wherein said method comprises contacting said cell, tissue, or organ with a solution that increases oxidation of a lipid; increases export of a lipid from said cell, tissue, or organ; or both.

7. The method of claim 1, wherein said intracellular lipid storage material is a triglyceride, a cholesterol, a cholesterol ester, or a phospholipid.

8. The method of claim 1, wherein said method results in reducing an ischemia-reperfusion injury in said cell, tissue, or organ upon transplantation into a recipient.

9. The method of claim 1, wherein said method results in reducing a cold-preservation-related injury in said cell, tissue, or organ upon transplantation into a recipient.

10. The method of claim 1, wherein said method reconditions a steatotic cell, tissue, or organ.

11. The method of claim 10, wherein said steatotic cell is a liver cell, said steatotic tissue is liver tissue, or said steatotic organ is a liver.

12. The method of claim 1, further comprising inducing heat shock of said cell, tissue, or organ.

13. The method of claim 12, wherein said inducing is the result of increasing the temperature of said cell, tissue, or organ by at least 1° C. for at least one minute.

14. The method of claim 13, wherein said temperature is increased for a period ranging between one minute and one hour.

15. (canceled)

16. (canceled)

17. The method of claim 13, wherein said temperature of said cell, tissue, or organ is increased to a range between 37° C. and 50° C.

18-20. (canceled)

21. The method of claim 13, wherein said increasing of said temperature is the result of heating the whole body of the donor of said cell, tissue, or organ.

22. The method of claim 13, wherein said increasing of said temperature is the result of heating a localized area of the donor including said cell, tissue, or organ.

23. The method of claim 13, wherein said heating is mediated by microwave or ultrasound treatment.

24. The method of claim 22, wherein said heating is mediated by warming the blood percolating said localized area.

25. The method of claim 13, wherein said increasing of said temperature is the result of heating said cell, tissue, or organ ex vivo.

26. The method of claim 12, wherein said inducing is the result of contacting said cell, tissue, or organ with an agent that increases the expression of at least one heat shock protein in said cell, tissue, or organ.

27. The method of claim 26, wherein said agent is cobalt protoporphyrin or geranylgeranylacetone.

28. The method of claim 26, wherein said cell, tissue, or organ is provided with at least one expression vector comprising a nucleic acid sequence encoding a heat shock protein.

29. The method of claim 1, further comprising administering a heat shock protein to said cell, tissue, or organ.

30. The method of claim 26, wherein said heat shock protein is selected from the group consisting of HSP72, HSP70, HO-1, and HSP90.

31-35. (canceled)

36. The method of claim 1, further comprising contacting said cell, tissue, or organ with a composition comprising gadolinium chloride (GdCl₃).

37. The method of claim 1, further comprising contacting said cell, tissue, or organ with a composition comprising an agent that inhibits the proliferation, activation, or both of T cells.

38. The method of claim 37, wherein said agent is selected from the group consisting of cyclosporine A (CyA) and FK506.

39-42. (canceled)

43. A solution for reducing intracellular lipid storage material of a donor cell, tissue, or organ comprising a catabolic hormone and an amino acid, wherein said catabolic hormone is selected from the group consisting of glucagon, epinephrine, growth hormone, hepatocyte growth factor, leptin, adiponectin, metformin, thyroid hormone, and a glucocorticoid hormone and wherein said amino acid is selected from the group consisting of alanine and glutamine.

44-62. (canceled)

63. A method for preparing a donor cell, tissue, or organ for transplantation into a recipient, said method comprising contacting said donor cell, tissue, or organ with the solution of claim 43.

64-87. (canceled)
